(19) United States
(12) Patent Application Publication
(10) Pub. No.: US 2015/0088193 A1
(43) Pub. Date: Mar. 26, 2015

(54) MEMBRANE REMOVING FORCEPS

(71) Applicants: Gregg D. Scheller, Wildwood, MO (US); Steven J. Apperson, Ellisville, MO (US)

(72) Inventors: Gregg D. Scheller, Wildwood, MO (US); Steven J. Apperson, Ellisville, MO (US); Justin M. Raney, O'Fallon, MO (US)

(73) Assignee: Katalyst Surgical, LLC, Chesterfield, MO (US)

(21) Appl. No.: 14/479,462

(22) Filed: Sep. 8, 2014

Related U.S. Application Data
(60) Provisional application No. 61/881,620, filed on Sep. 24, 2013.

(51) Int. Cl. A61F 9/007; A61B 17/29

(52) U.S. Cl. CPC A61F 9/00736; A61B 17/2909; A61B 17/29; A61B 2017/2926
USPC 606/207

(57) ABSTRACT

A membrane removing forceps may include a plurality of forceps jaws and a plurality of membrane hooks. Each membrane hook may have a membrane hook outer height. Each membrane hook outer height may be less than the average thickness of a membrane, e.g., each membrane hook outer height may be less than the average thickness of an internal limiting membrane. A surgeon may pierce a membrane wherein only the membrane hooks penetrate the membrane, e.g., a surgeon may pierce an internal limiting membrane wherein only the membrane hooks penetrate the internal limiting membrane. The surgeon may then grasp and remove the membrane without damaging an underlying tissue, e.g., the surgeon may then grasp and remove the internal limiting membrane without damaging an underlying retina.

### CROSS-REFERENCE TO RELATED APPLICATIONS

This application claims the benefit of U.S. Provisional Application No. 61/881,620, filed Sep. 24, 2013.

### FIELD OF THE INVENTION

The present disclosure relates to a surgical instrument, and, more particularly, to a microsurgical forceps.

### BACKGROUND OF THE INVENTION

A microsurgical forceps may be used to perform a microsurgical procedure, e.g., an ophthalmic surgical procedure. For example, a surgeon may use a forceps to grasp and manipulate tissues or other surgical instruments to perform portions of a surgical procedure. A particular microsurgical procedure may require a surgeon to separate a first tissue from a second tissue without causing trauma to at least one of the tissues. Such a separation procedure may be particularly difficult for a surgeon to perform if the tissue surface geometry is not flat, e.g., if the tissue surface geometry is convex. For example, an ophthalmic surgeon may be required to remove an internal limiting membrane from a patient's retina without causing trauma to the patient's retina. Accordingly, there is a need for a microsurgical forceps that enables a surgeon to separate a first tissue from a second tissue without causing significant trauma to at least one of the tissues.

### BRIEF SUMMARY OF THE INVENTION

Illustratively, a membrane removing forceps may comprise a plurality of forceps jaws and a plurality of membrane hooks. In one or more embodiments, each membrane hook may comprise a membrane hook outer height. Illustratively, each membrane hook outer height may be less than an average thickness of a membrane, e.g., each membrane hook outer height may be less than the average thickness of an internal limiting membrane. In one or more embodiments, a surgeon may pierce a membrane wherein only the membrane hooks penetrate the membrane, e.g., a surgeon may pierce an internal limiting membrane wherein only the membrane hooks penetrate the internal limiting membrane.

Illustratively, the surgeon may then grasp and remove the membrane without damaging an underlying tissue, e.g., the surgeon may then grasp and remove the internal limiting membrane without damaging an underlying retina.

### BRIEF DESCRIPTION OF THE DRAWINGS

The above and further advantages of the present invention may be better understood by referring to the following description in conjunction with the accompanying drawings in which like reference numerals indicate identical or functionally similar elements:

FIG. 1 is a schematic diagram illustrating an exploded view of a surgical instrument assembly;

FIGS. 2A and 2B are schematic diagrams illustrating an assembled surgical instrument;

FIG. 3 is a schematic diagram illustrating a membrane removing forceps;

FIGS. 4A, 4B, and 4C are schematic diagrams illustrating a gradual closing of a membrane removing forceps;

FIGS. 5A, 5B, and 5C are schematic diagrams illustrating a gradual opening of a membrane removing forceps;

FIGS. 6A, 6B, 6C, 6D, 6E and 6F are schematic diagrams illustrating a membrane removal;

FIG. 7 is a schematic diagram illustrating a membrane removing forceps;

FIGS. 8A, 8B, and 8C are schematic diagrams illustrating a gradual closing of a membrane removing forceps;

FIGS. 9A, 9B, and 9C are schematic diagrams illustrating a gradual opening of a membrane removing forceps;

FIGS. 10A, 10B, 10C, 10D, 10E and 10F are schematic diagrams illustrating a membrane removal.

### DETAILED DESCRIPTION OF AN ILLUSTRATIVE EMBODIMENT

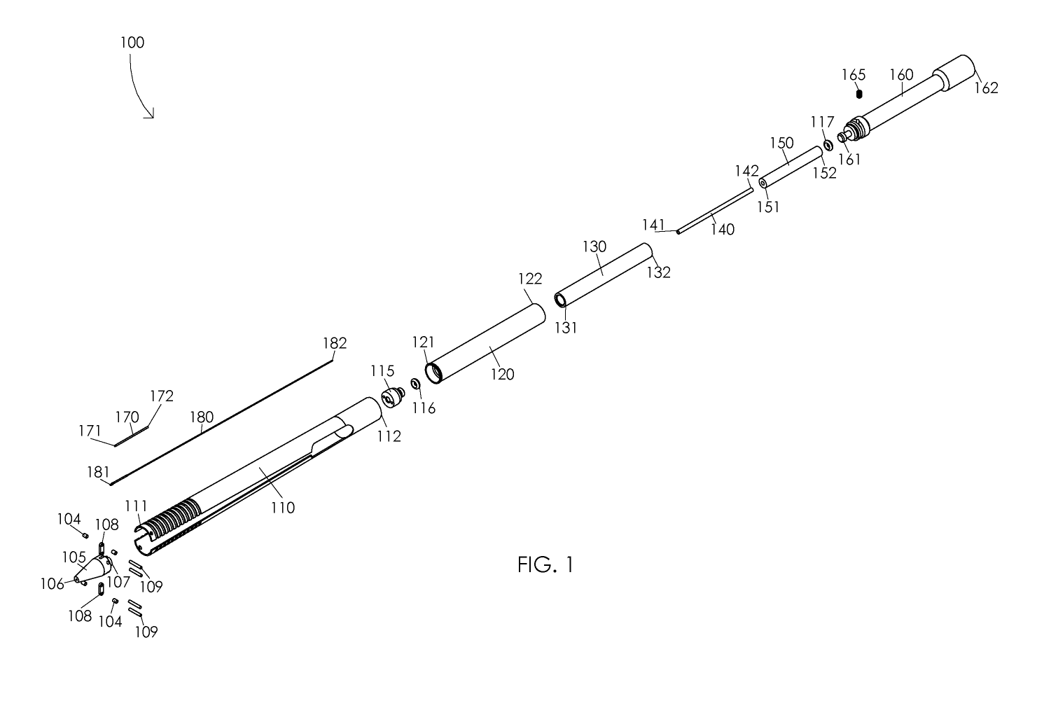

FIG. 1 is a schematic diagram illustrating an exploded view of a surgical instrument assembly 100. In one or more embodiments, surgical instrument assembly 100 may comprise a nosecone 105 having a nosecone distal end 106 and a nosecone proximal end 107; one or more links 108; one or more link pins 109; one or more spacers 104; a handle 110 having a handle distal end 111 and a handle proximal end 112; a front plug 115; a distal O-ring 116; a proximal O-ring 117; a housing sleeve 120 having a housing sleeve distal end 121 and a housing sleeve proximal end 122; an actuation facilitating sleeve 130 having an actuation facilitating sleeve distal end 131 and an actuation facilitating sleeve proximal end 132; an inner hypodermic tube 140 having an inner hypodermic tube distal end 141 and an inner hypodermic tube proximal end 142; a piston tube 150 having a piston tube distal end 151 and a piston tube proximal end 152; an end plug 160 having an end plug distal end 161 and an end plug proximal end 162; a fixation mechanism 165; an outer hypodermic tube 170 having an outer hypodermic tube distal end 171 and an outer hypodermic tube proximal end 172; and a surgical blank 180 having a surgical blank distal end 181 and a surgical blank proximal end 182.

Illustratively, outer hypodermic tube 170 may be fixed to nosecone 105, e.g., outer hypodermic tube proximal end 172 may be fixed to nosecone distal end 106. In one or more embodiments, one or more links 108 and one or more link pins 109 may be configured to connect nosecone 105 and handle 110, e.g., a portion of nosecone 105 may be disposed within handle 110. Illustratively, nosecone 105 may be connected to one or more links 108, e.g., one or more link pins 109 may be disposed within both nosecone 105 and one or more links 108. In one or more embodiments, handle 110 may be connected to one or more links 108, e.g., one or more link pins 109 may be disposed within both handle 110 and one or more links 108. Illustratively, at least one link 108 may be connected to both nosecone 105 and handle 110, e.g., by one or more link pins 109.

In one or more embodiments, inner hypodermic tube 140 may be at least partially disposed within piston tube 150, e.g., inner hypodermic tube proximal end 142 may be disposed within piston tube 150. Illustratively, inner hypodermic tube 140 and piston tube 150 may be at least partially disposed within actuation facilitating sleeve 130. In one or more embodiments, actuation facilitating sleeve 130 and piston tube 150 may be disposed within housing sleeve 120. Illustratively, inner hypodermic tube 140 may be at least partially disposed within housing sleeve 120, e.g., inner hypodermic tube distal end 141 may extend a distance from housing sleeve distal end 121.

In one or more embodiments, distal O-ring 116 may be disposed over a portion of front plug 115. Illustratively, distal O-ring 116 may be disposed within housing sleeve 120 and actuation facilitating sleeve 130. In one or more embodiments, at least a portion of front plug 115 may be disposed within housing sleeve 120 and actuation facilitating sleeve 130, e.g., housing sleeve distal end 121 and actuation facilitating sleeve distal end 131 may be disposed over a portion of front plug 115. Illustratively, proximal O-ring 117 may be disposed over a portion of end plug 160. In one or more embodiments, proximal O-ring 117 may be disposed within housing sleeve 120 and actuation facilitating sleeve 130. Illustratively, at least a portion of end plug 160 may be disposed within housing sleeve 120 and actuation facilitating sleeve 130, e.g., housing sleeve proximal end 122 and actuation facilitating sleeve proximal end 132 may be disposed over a portion of end plug 160.

In one or more embodiments, front plug 115, distal O-ring 116, housing sleeve 120, actuation facilitating sleeve 130, piston tube 150, inner hypodermic tube 140, proximal O-ring 117, and end plug 160 may be disposed within handle 110. For example, end plug 160 may be disposed within handle 110 wherein end plug proximal end 162 may be adjacent to handle proximal end 112. Illustratively, inner hypodermic tube 140 may be fixed to nosecone 105, e.g., inner hypodermic tube distal end 141 may be fixed to nosecone proximal end 107.

In one or more embodiments, surgical blank 180 may be disposed within outer hypodermic tube 170, nosecone 105, inner hypodermic tube 140, piston tube 150, and end plug 160. Illustratively, fixation mechanism 165 may be configured to fix surgical blank 180 in a position relative to handle 110. For example, fixation mechanism 165 may comprise a setscrew configured to fix surgical blank 180 in a position relative to handle 110. In one or more embodiments, fixation mechanism 165 may comprise an adhesive material configured to fix surgical blank 180 in a position relative to handle 110. Illustratively, fixation mechanism 165 may comprise any suitable means of fixing surgical blank 180 in a position relative to handle 110.

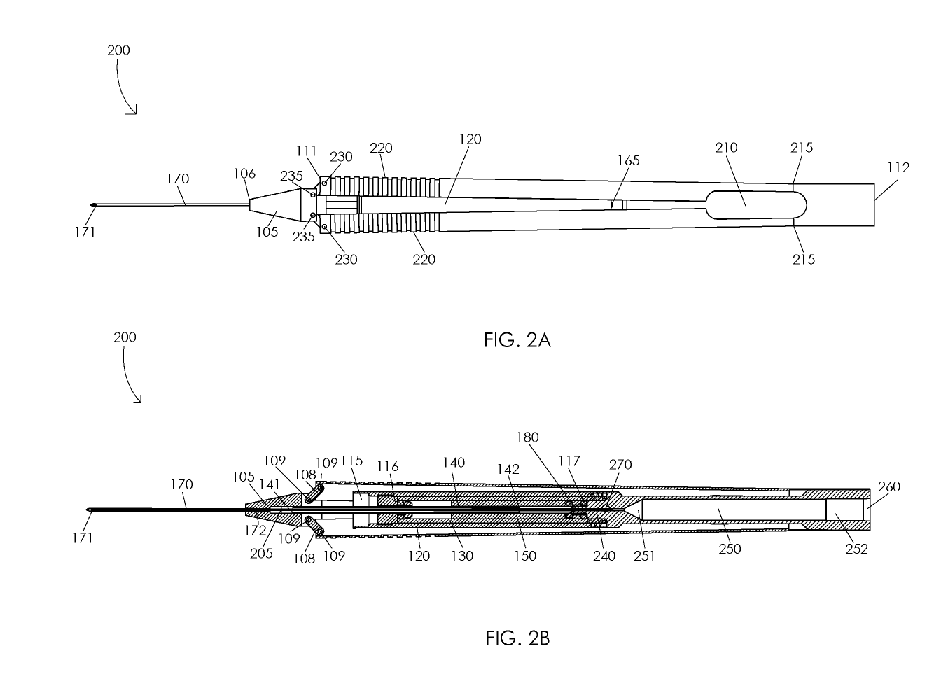

FIGS. 2A and 2B are schematic diagrams illustrating an assembled surgical instrument 200. FIG. 2A illustrates a side view of an assembled surgical instrument 200. In one or more embodiments, housing sleeve 120 may be disposed within handle 110. Illustratively, actuation facilitating sleeve 130 may be disposed within housing sleeve 120. In one or more embodiments, piston tube 150 may be disposed within actuation facilitating sleeve 130. Illustratively, a portion of inner hypodermic tube 140 may be disposed within piston tube 150, e.g., inner hypodermic tube proximal end 142 may be disposed within piston tube 150. In one or more embodiments, a portion of inner hypodermic tube 140 may be fixed to an inner portion of piston tube 150, e.g., by a biocompatible adhesive. For example, an actuation of inner hypodermic tube 140 relative to handle 110 may be configured to actuate piston tube 150 relative to handle 110 and an actuation of piston tube 150 relative to handle 110 may be configured to actuate inner hypodermic tube 140 relative to handle 110.

Illustratively, handle 110 may comprise a spring return aperture 210. In one or more embodiments, spring return aperture 210 may comprise one or more hinges 215. Illustratively, spring return aperture 210 may be configured to separate a first portion of handle 110 and a second portion of handle 110. In one or more embodiments, spring return aperture 210 may be configured to separate a particular point on the first portion of handle 110 from a particular point on the second portion of handle 110 at a first distance. Illustratively, an application of a compressive force to a portion of handle 110 may be configured to separate the particular point on the first portion of handle 110 from the particular point on the second portion of handle 110 at a second distance. In one or more embodiments, the first distance may be greater than the second distance.

Illustratively, handle 110 may comprise one or more surgical grip points 220. In one or more embodiments, one or more surgical grip points 220 may be configured to prevent undesirable movements of handle 110, e.g., during a surgical procedure. Illustratively, one or more surgical grip points 220 may be configured to interface with a surgeon's fingertips. In one or more embodiments, one or more surgical grip points 220 may be configured to increase a total contact area between a surgeon's fingertips and handle 110. Illustratively, one or more surgical grip points 220 may be configured to facilitate an application of a compressive force to handle 110, e.g., by increasing a coefficient of friction between a surgeon's fingertips and handle 110 as the surgeon applies a compressive force to handle 110. Handle 110 may be manufactured from any suitable material, e.g., polymers, metals, metal alloys, etc., or from any combination of suitable materials.

In one or more embodiments, handle 110 may comprise one or more handle link pin housings 230. Illustratively, handle link pin housing 230 may be configured to house link pin 109. In one or more embodiments, nosecone 105 may comprise one or more nosecone link pin housings 235. Illustratively, nosecone link pin housing 235 may be configured to house link pin 109. In one or more embodiments, at least one link pin 109 may be configured to connect nosecone 105 to link 108, e.g., link pin 109 may be disposed within both nosecone link pin housing 235 and link 108. Illustratively, at least one link pin 109 may be configured to connect handle 110 and link 108, e.g., link pin 109 may be disposed within both handle link pin housing 230 and link 108. In one or more embodiments, at least one link 108 may be connected to both nosecone 105 and handle 110, e.g., at least one link pin 109 may be disposed within both nosecone link pin housing 235 and link 108 and at least one link pin 109 may be disposed within both handle link pin housing 230 and link 108.

FIG. 2B illustrates a cross-sectional view of an assembled surgical instrument 200. In one or more embodiments, nosecone 105 may comprise a nosecone inner bore 205. Illustratively, inner hypodermic tube distal end 141 may be fixed within nosecone inner bore 205, e.g., by a machine press fit, a biocompatible adhesive, etc. In one or more embodiments, outer nosecone proximal end 172 may be fixed within nosecone inner bore 205, e.g., by a machine press fit, a biocompatible adhesive, etc.

Illustratively, end plug 160 may comprise a surgical blank housing 240, an end plug inner bore 250, an interface taper 260, and a fixation mechanism housing 270. In one or more embodiments, end plug inner bore 250 may comprise an end plug inner bore distal cone 251 and an end plug inner bore proximal chamber 252. Illustratively, interface taper 260 may be configured to interface with one or more components, e.g., to provide one or more surgical utilities. In one or more embodiments, interface taper 260 may comprise a Luer taper. End plug 160 may be manufactured from any suitable material, e.g., polymers, metals, metal alloys, etc., or from any combination of suitable materials.

Illustratively, surgical blank 180 may be disposed within outer hypodermic tube 170, nosecone inner bore 205, inner hypodermic tube 140, piston tube 150, actuation facilitating sleeve 130, surgical blank housing 240, and fixation mechanism housing 270. In one or more embodiments, fixation mechanism 165 may be configured to fix surgical blank 180 in a position relative to handle 110, e.g., at fixation mechanism housing 270. For example, fixation mechanism 165 may be disposed within fixation mechanism houses ing 270, e.g., to fix surgical blank 180 in a position relative to handle 110.

Illustratively, surgical blank 180 may modified to provide a one or more surgical utilities, e.g., surgical blank distal end 181 may be modified to provide one or more particular surgical utilities of a plurality of surgical utilities. In one or more embodiments, surgical blank 180 may be modified wherein surgical blank 180 may comprise a surgical forceps, e.g., with a grasping utility. Illustratively, surgical blank 180 may be modified wherein surgical blank 180 may comprise a surgical scissors, e.g., with a cutting utility. In one or more embodiments, surgical blank 180 may be modified wherein surgical blank 180 may comprise a surgical manipulator, e.g., with a manipulation utility. Illustratively, surgical blank 180 may be modified wherein surgical blank 180 may comprise a surgical hook, e.g., with a hook utility. In one or more embodiments, surgical blank 180 may be modified wherein surgical blank 180 may comprise a surgical chopper, e.g. with a chopping utility. Illustratively, surgical blank 180 may be modified wherein surgical blank 180 may comprise a surgical pre-chopper, e.g., with a pre-chopping utility. In one or more embodiments, surgical blank 180 may be modified wherein surgical blank 180 may comprise a surgical pick, e.g., with a pick utility. Illustratively, surgical blank 180 may be modified to comprise any surgical instrument with any surgical utility as will be appreciated by one having ordinary skill in the relevant technological art. Surgical blank 180 may be manufactured from any suitable material, e.g., polymers, metals, metal alloys, etc., or from any combination of suitable materials.

In one or more embodiments, handle 110 may be compressed, e.g., by an application of a compressive force to handle 110. For example, a surgeon may compress handle 110 by gently squeezing handle 110, e.g., at one or more surgical grip points 220. Illustratively, a compression of handle 110 may be configured to actuate nosecone 105 relative to handle proximal end 112. Illustratively, a compression of handle 110 may be configured to extend nosecone 105 relative to handle proximal end 112.

In one or more embodiments, a compression of handle 110 may be configured to extend one or more links 108 connected to nosecone 105, e.g., by one or more link pins 109, away from handle proximal end 112. Illustratively, a compression of handle 110 may be configured to gradually project nosecone 105 relative to handle proximal end 112. In one or more embodiments, a compression of handle 110 may be configured to gradually actuate outer hypodermic tube 170 relative to handle proximal end 112. For example, a compression of handle 110 may be configured to gradually extend outer hypodermic tube 170 relative to handle proximal end 112. Illustratively, a compression of handle 110 may be configured to gradually actuate outer hypodermic tube 170 relative to surgical blank 180. For example, a compression of handle 110 may be configured to gradually extend outer hypodermic tube 170 relative to surgical blank 180.

In one or more embodiments, a compression of handle 110 may be configured to actuate inner hypodermic tube 140 relative to handle 110. Illustratively, a compression of handle 110 may be configured to extend inner hypodermic tube 140 relative to handle proximal end 112. In one or more embodiments, a compression of handle 110 may be configured to actuate piston tube 150 relative to handle 110. Illustratively, a compression of handle 110 may be configured to extend piston tube 150 relative to handle proximal end 112.

In one or more embodiments, handle 110 may be decompressed, e.g., by reducing a magnitude of a compressive force applied to handle 110. For example, a surgeon may decompress handle 110 by decreasing an amount of compressive force applied to handle 110, e.g., at one or more surgical grip points 220. Illustratively, a decompression of handle 110 may be configured to actuate nosecone 105 relative to handle proximal end 112. Illustratively, a decompression of handle 110 may be configured to retract nosecone 105 relative to handle proximal end 112.

In one or more embodiments, a decompression of handle 110 may be configured to retract one or more links 108 connected to nosecone 105, e.g., by one or more link pins 109, towards handle proximal end 112. Illustratively, a decompression of handle 110 may be configured to gradually retract nosecone 105 relative to handle proximal end 112. In one or more embodiments, a decompression of handle 110 may be configured to gradually actuate outer hypodermic tube 170 relative to handle proximal end 112. For example, a decompression of handle 110 may be configured to gradually retract outer hypodermic tube 170 relative to handle proximal end 112. Illustratively, a decompression of handle 110 may be configured to gradually actuate outer hypodermic tube 170 relative to surgical blank 180. For example, a decompression of handle 110 may be configured to gradually retract outer hypodermic tube 170 relative to surgical blank 180.

In one or more embodiments, a decompression of handle 110 may be configured to actuate inner hypodermic tube 140 relative to handle 110. Illustratively, a decompression of handle 110 may be configured to retract inner hypodermic tube 140 relative to handle proximal end 112. In one or more embodiments, a decompression of handle 110 may be configured to actuate piston tube 150 relative to handle 110. Illustratively, a decompression of handle 110 may be configured to retract piston tube 150 relative to handle proximal end 112.

In one or more embodiments, actuation facilitating sleeve 130 and piston tube 150 may be configured to minimize a coefficient of friction between actuation facilitating sleeve 130 and piston tube 150. Illustratively, actuation facilitating sleeve 130 and piston tube 150 may be manufactured from one or more materials configured to minimize a friction force, e.g., when piston tube 150 is actuated relative to handle 110. For example, actuation facilitation sleeve 130 and piston tube 150 may be manufactured from one or more materials configured to minimize a friction force, e.g., when piston tube 150 is actuated relative to actuation facilitating sleeve 130. In one or more embodiments, at least an inner portion of actuation facilitating sleeve 130 may comprise a non-crystalline material, e.g., glass. Illustratively, at least an outer portion of piston tube 150 may comprise carbon or a carbon allotrope, e.g., graphite. In one or more embodiments, at least an inner portion of actuation facilitating sleeve 130 may comprise a carbon or a carbon allotrope, e.g., graphite. Illustratively, at least an outer portion of piston tube 150 may comprise a non-crystalline material, e.g., glass.

Actuation facilitating sleeve 130 may be manufactured from any suitable material, e.g., polymers, metals, metal alloys, etc., or from any combination of suitable materials. Piston tube 150 may be manufactured from any suitable material, e.g., polymers, metals, metal alloys, etc., or from any combination of suitable materials. In one or more embodiments, an inner portion of actuation facilitating sleeve 130 may be coated with a material configured to minimize a coefficient of friction between actuation facilitating sleeve 130 and piston tube 150, e.g., Teflon. Illustratively, an outer portion of piston tube 150 may be coated with a material configured to minimize a coefficient of friction between piston tube 150 and actuation facilitation sleeve 130, e.g., Teflon.

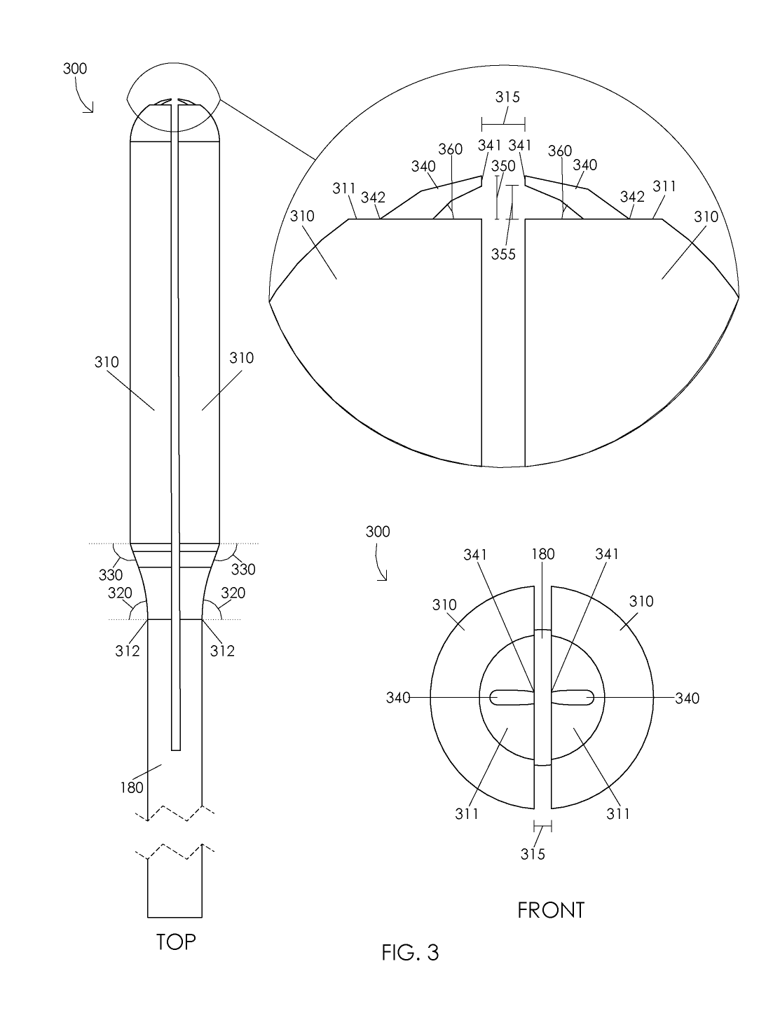

FIG. 3 is a schematic diagram illustrating a membrane removing forceps 300. FIG. 3 illustrates a top view and a front view of a membrane removing forceps 300. Illustratively, membrane removing forceps 300 may be manufactured with dimensions configured for performing microsurgical procedures, e.g., ophthalmic surgical procedures. In one or more embodiments, membrane removing forceps 300 may be manufactured from surgical blank 180. In one or more embodiments, membrane removing forceps 300 may be manufactured by modifying surgical blank 180, e.g., with an electric discharge machine, a laser, a file, deep reactive ion etching, or any suitable modification means. Illustratively, membrane removing forceps 300 may comprise a plurality of forceps jaws 310 wherein each forceps jaw 310 has a forceps jaw distal end 311 and a forceps jaw proximal end 312. In one or more embodiments, each forceps jaw distal end 311 may have a surface area in a range of 0.03 to 0.15 square millimeters, e.g., each forceps jaw distal end 311 may have a surface area of 0.065 square millimeters. Illustratively, each forceps jaw distal end 311 may have a surface area less than 0.03 square millimeters or greater than 0.15 square millimeters. In one or more embodiments, forceps jaws distal ends 311 may be separated by a forceps jaw maximum separation distance 315. Illustratively, forceps jaw maximum separation distance 315 may comprise a distance in a range of 50.0 to 800.0 micrometers, e.g., forceps jaw maximum separation distance 315 may comprise a distance of 600 micrometers. In one or more embodiments, a geometry of membrane removing forceps 300 may comprise a first contour angle 320 and a second contour angle 330. Illustratively, first contour angle 320 may comprise any angle less than or equal to 90.0 degrees, e.g., first contour angle 320 may comprise an 80.0 degree angle. In one or more embodiments, first contour angle 320 may comprise an angle in a range of 60.0 to 80.0 degrees, e.g., first contour angle 320 may comprise a 72.3 degree angle. Illustratively, first contour angle 320 may comprise an angle less than 60.0 degrees or greater than 80.0 degrees. In one or more embodiments, second contour angle 330 may comprise any angle greater than or equal to 90.0 degrees, e.g., second contour angle 330 may comprise a 100.0 degree angle. Illustratively, second contour angle 330 may comprise an angle in a range of 95.0 to 120.0 degrees, e.g., second contour angle 330 may comprise a 107.0 degree angle. In one or more embodiments, second contour angle 330 may comprise an angle less than 95.0 degrees or greater than 120.0 degrees.

Illustratively, membrane removing forceps 300 may comprise a plurality of membrane hooks 340 wherein each membrane hook 340 has a membrane hook distal end 341 and a membrane hook proximal end 342. In one or more embodiments, each membrane hook 340 extends from a forceps jaw distal end 311, e.g., forceps jaw distal end 311 may be adjacent to membrane hook proximal end 342. For example, a first membrane hook 340 may extend from a first forceps jaw distal end 311 and a second membrane hook 340 may extend from a second forceps jaw distal end 311. Illustratively, membrane hook 340 may be configured to grasp a portion of a membrane, e.g., membrane hook 340 may be configured to grasp a portion of an internal limiting membrane 670. In one or more embodiments, membrane hook 340 may be configured to grasp a portion of a first tissue disposed over a second tissue without damaging the second tissue. Illustratively, membrane hook 340 may be configured to grasp a first tissue having a convex surface geometry disposed over a second tissue having a convex surface geometry without damaging the second tissue. In one or more embodiments, each membrane hook 340 may have a surface area in a range of 25.0 to 75.0 square micrometers, e.g., each membrane hook 340 may have a surface area of 48.7 square micrometers. Illustratively, each membrane hook 340 may have a surface area less than 25.0 square micrometers or greater than 75.0 square micrometers.

Illustratively, each membrane hook 340 may comprise a membrane hook outer height 350, a membrane hook inner height 355, and a membrane hook angle 360. In one or more embodiments, membrane hook angle 360 may comprise any angle less than 90.0 degrees, e.g., membrane hook angle 360 may comprise a 45.0 degree angle. Illustratively, membrane hook angle 360 may comprise an angle in a range of 5.0 to 20.0 degrees, e.g., membrane hook angle 360 may comprise a 10.0 degree angle. In one or more embodiments, membrane hook angle 360 may comprise an angle less than 5.0 degrees or greater than 20.0 degrees, e.g., membrane hook angle 360 may comprise a 3.8 degree angle. Illustratively, membrane hook outer height 350 may be configured to prevent damage to a tissue underlying a membrane, e.g., membrane hook outer height 350 may be configured to prevent damage to a retina 660 underlying an internal limiting membrane 670. In one or more embodiments, membrane hook outer height 350 may comprise a distance that is a fraction of an average membrane thickness, e.g., membrane hook outer height 350 may comprise a distance that is a fraction of an average internal limiting membrane thickness. Illustratively, membrane hook outer height 350 may comprise a distance in a range of 0.25 to 3.0 micrometers, e.g., membrane hook outer height 350 may comprise a distance of 1.25 micrometers. In one or more embodiments, membrane hook outer height 350 may comprise a distance less than 0.25 micrometers or greater than 3.0 micrometers, e.g., membrane hook outer height 350 may comprise a distance of 0.15 micrometers. Illustratively, a particular membrane hook outer height 350 may be selected, e.g., by a surgeon, on a case-by-case basis. For example, a surgeon may select a first membrane removing forceps 300 having a first membrane hook outer height 350 to remove a first membrane and the surgeon may select a second membrane removing forceps 300 having a second membrane hook outer height 350 to remove a second membrane wherein the first membrane is thicker than the second membrane and the first membrane hook outer height 350 is greater than the second membrane hook outer height 350. Illustratively, membrane hook inner height 355 may be configured to grasp a portion of a membrane, e.g., membrane hook inner height 355 may be configured to grasp a portion of an internal limiting membrane 670. In one or more embodiments, membrane hook inner height 355 may comprise any distance less than membrane hook outer height 350, e.g., membrane hook inner height 355 may comprise a distance equal to 80.0 percent of membrane hook outer height 350. Illustratively, membrane hook inner height 355 may comprise a distance in a range of 0.1 to 2.9 micrometers, e.g., membrane hook inner height 355 may comprise a distance of 1.0 micrometers. In one or more embodiments, membrane hook inner height 355 may comprise a distance less than 0.1 micrometers or greater than 2.9 micrometers, e.g., membrane hook inner height 355 may comprise a distance of 0.05 micrometers.

Illustratively, membrane hook 340 may be manufactured by modifying surgical blank 180, e.g., by laser ablation. In one or more embodiments, membrane hook 340 may be manufactured by modifying surgical blank 180, e.g., by femtosecond laser ablation. Illustratively, membrane hook 340 may be manufactured by laser ablation of surgical blank 180 in multiple orientations. In one or more embodiments, membrane hook 340 may be manufactured by performing laser ablation of surgical blank 180 in a first orientation, e.g., and then performing laser ablation of surgical blank 180 in a second orientation. Illustratively, membrane hook 340 may be manufactured by performing a first laser ablation of surgical blank 180 and then rotating surgical blank 180 to perform a second laser ablation of surgical blank 180. In one or more embodiments, membrane hook 340 may be manufactured by performing a first laser ablation of surgical blank 180 and then rotating surgical blank 180 by 90.0 degrees to perform a second laser ablation of surgical blank 180. Illustratively, membrane hook 340 may be formed by high precision micromachining using a 355 nm Nd: vanadate laser operating at 10 kHz and with an average power of 7.0 Watts and pulse duration of 35.0 nanoseconds. In one or more embodiments, membrane hook 340 may be manufactured by using an electric discharge machine to shape membrane removing forceps 300 from blank 180 and then using laser micromachining to shape membrane hook 340. Illustratively, membrane hook 340 may be manufactured by modifying surgical blank 180, e.g., by deep reactive-ion etching. In one or more embodiments, membrane hook 340 may be manufactured by modifying surgical blank 180, e.g., by the Bosch process of time-multiplexed etching. Illustratively, membrave hook 340 may be manufactured by exposing a portion of surgical blank 180 to repeated cycles of isotropic plasma etching followed by deposition of a chemically inert passivation layer. In one or more embodiments, membrane hook 340 may be manufactured by fabricating a membrane hook 340 on a substrate and then fixing the substrate to a portion of surgical blank 180. Illustratively, surgical blank 180 may be modified, e.g., by deep reactive-ion etching, to manufacture membrane hook 340. In one or more embodiments, surgical blank 180 may be modified wherein one or more portions of surgical blank 180 comprise membrane hook 340 and then surgical blank 180 may be modified to manufacture membrane removing forceps 300. Illustratively, membrane hook 340 may be manufactured by deep reactive-ion etching of surgical blank 180 in multiple orientations. In one or more embodiments, membrane hook 340 may be manufactured by performing deep reactive-ion etching of surgical blank 180 in a first orientation, e.g., and then performing deep reactive-ion etching of surgical blank 180 in a second orientation. Illustratively, membrane hook 340 may be manufactured by performing a first deep reactive-ion etching of surgical blank 180 and then rotating surgical blank 180 to perform a second deep reactive-ion etching of surgical blank 180. In one or more embodiments, membrane hook 340 may be manufactured by performing a first deep reactive-ion etching of surgical blank 180 and then rotating surgical blank 180 by 90.0 degrees to perform a second deep reactive-ion etching of surgical blank 180. Illustratively, surgical blank 180 may be modified, e.g., by an electric discharge machine, to manufacture membrane removing forceps 300 and then membrane removing forceps 300 may be modified, e.g., by deep reactive-ion etching, to fabricate membrane hook 340.

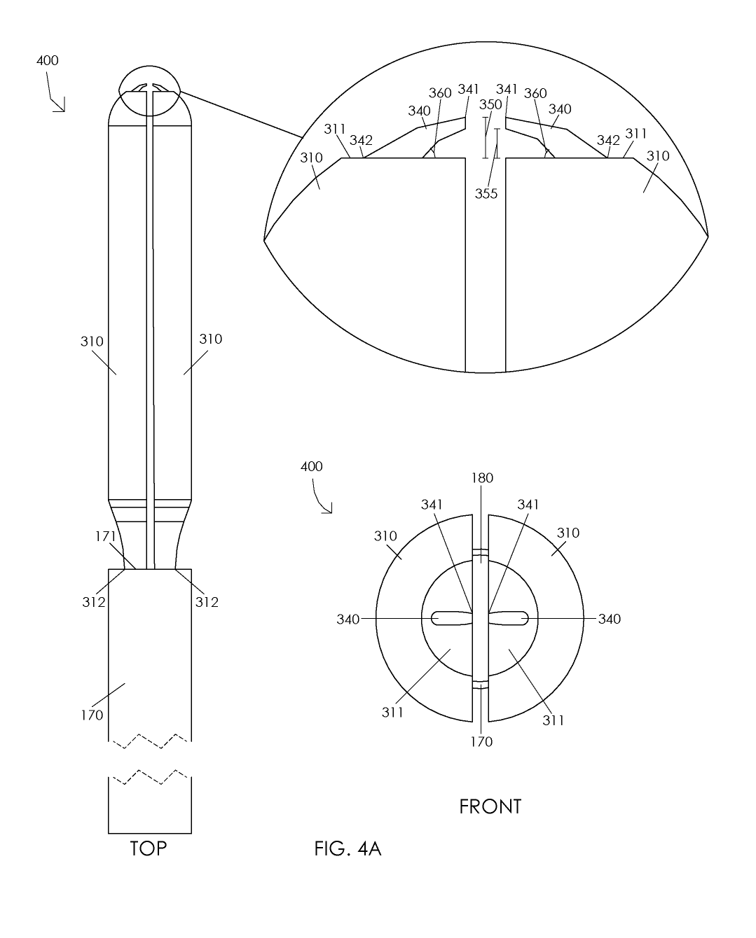

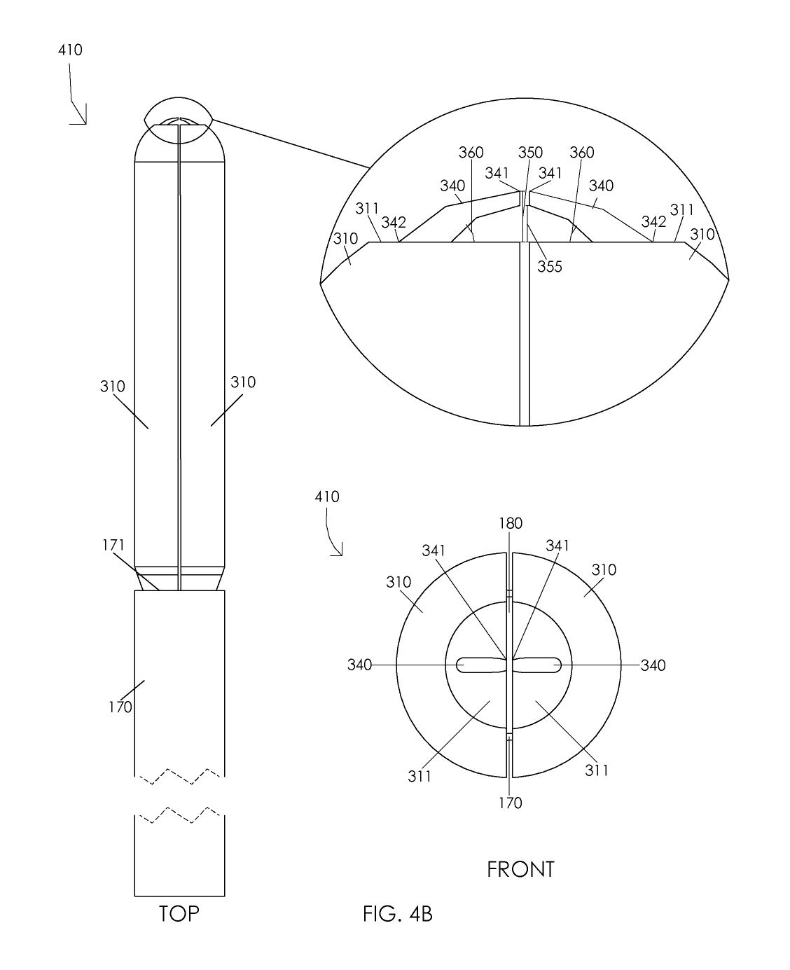

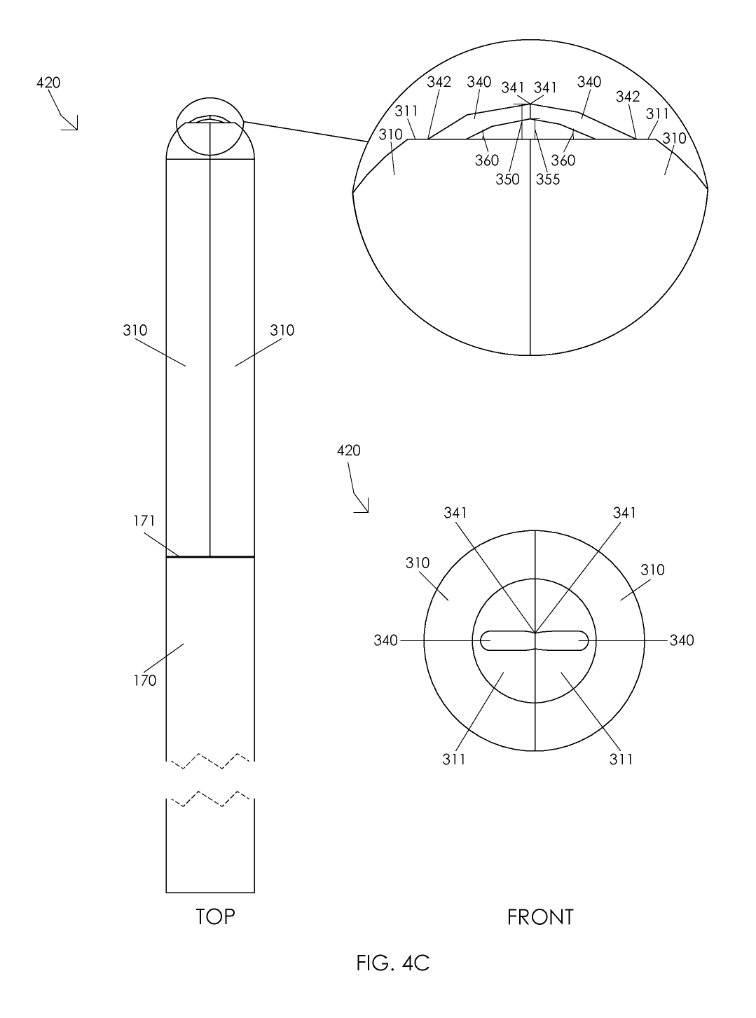

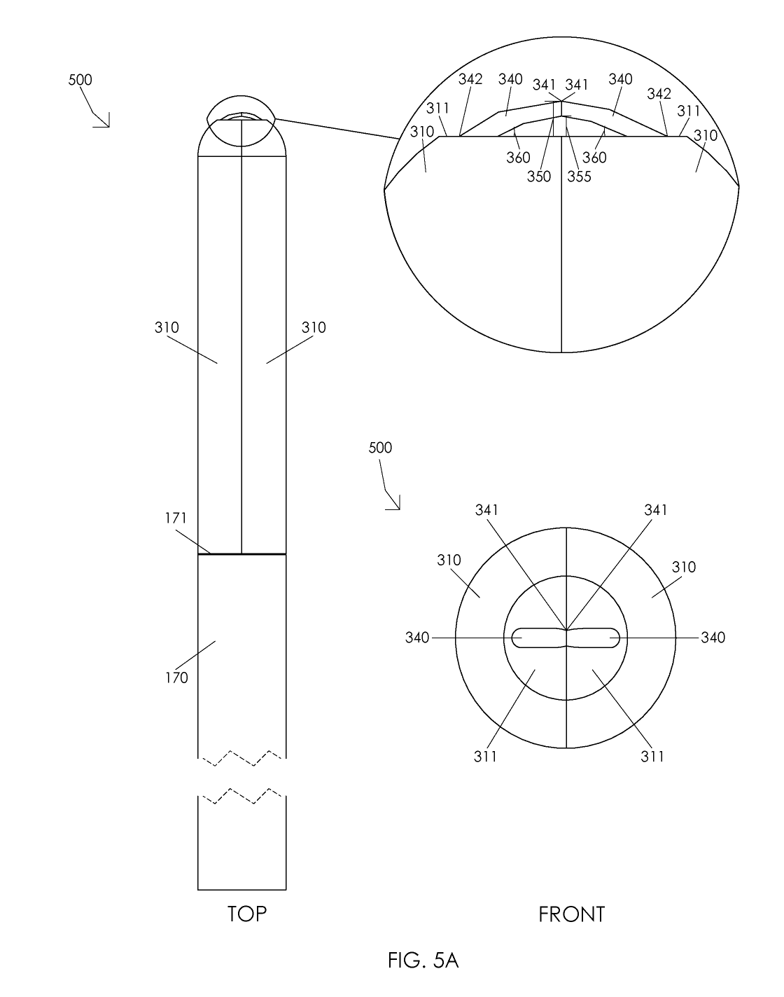

FIGS. 4A, 4B, and 4C are schematic diagrams illustrating a gradual closing of a membrane removing forceps 300. FIG. 4A illustrates a top view and a front view of an open membrane removing forceps 400. In one or more embodiments, membrane removing forceps 300 may comprise an open membrane removing forceps 400, e.g., when a first forceps jaw distal end 311 is separated from a second forceps jaw distal end 311 by forceps jaw maximum separation distance 315. Illustratively, membrane removing forceps 300 may comprise an open membrane removing forceps 400, e.g., when outer hypodermic tube 170 is fully retracted relative to forceps jaws proximal ends 312. Illustratively, membrane removing forceps 300 may comprise an open membrane removing forceps 400, e.g., when handle 110 is fully decompressed.

FIG. 4B illustrates a top view and a front view of a partially closed membrane removing forceps 410. In one or more embodiments, a compression of handle 110 may be configured to gradually close a membrane removing forceps 300, e.g., from an open membrane removing forceps 400 to a partially closed membrane removing forceps 410. Illustratively, a compression of handle 110 may be configured to extend outer hypodermic tube 170 relative to surgical blank 180, e.g., a compression of handle 110 may be configured to extend outer hypodermic tube distal end 171 over forceps jaws proximal ends 312. In one or more embodiments, a compression of handle 110 may be configured to decrease a distance between a first forceps jaw distal end 311 and a second forceps jaw distal end 311, e.g., a first forceps jaw distal end 311 and a second forceps jaw distal end 311 may be separated by a distance less than forceps jaw maximum separation distance 315 when membrane removing forceps 300 comprises a partially closed membrane removing forceps 410.

FIG. 4C illustrates a top view and a front view of a fully closed membrane removing forceps 420. Illustratively, a compression of handle 110 may be configured to gradually close a membrane removing forceps 300, e.g., from a partially closed membrane removing forceps 410 to a fully closed membrane removing forceps 420. In one or more embodiments, a compression of handle 110 may be configured to extend outer hypodermic tube 170 relative to surgical blank 180, e.g., a compression of handle 110 may be configured to extend outer hypodermic tube distal end 171 over forceps jaws proximal ends 312. Illustratively, an extension of outer hypodermic tube 170 over forceps jaws proximal ends 312 may be configured to close forceps jaws 310 wherein forceps jaws 310 initially contact at forceps jaws distal ends 311. In one or more embodiments, an extension of outer hypodermic tube 170 over forceps jaws proximal end 312 may be configured to close forceps jaws 310 wherein membrane hooks 340 initially contact at membrane hooks distal ends 341. Illustratively, a compression of handle 110 may be configured to gradually close forceps jaws 310 wherein forceps jaws 310 initially contact at forceps jaws distal ends 311. In one or more embodiments, a compression of handle 110 may be configured to gradually close forceps jaws 310 wherein membrane hooks 340 initially contact at membrane hooks distal ends 341. Illustratively, after forceps jaws distal ends 311 initially contact, a compression of handle 110 may be configured to gradually close forceps jaws 310 wherein a contact area between forceps jaws 310 gradually increases. In one or more embodiments, forceps jaws 310 may be configured to close wherein an amount of a first forceps jaw 310 in contact with a second forceps jaw 310 increases gradually from forceps jaws distal ends 311, e.g., forceps jaws 310 may be configured to close wherein an amount of a first forceps jaw 310 in contact with a second forceps jaw 310 increases gradually towards forceps jaws proximal ends 312. Illustratively, a compression of handle 110 may be configured to close forceps jaws 310 starting at forceps jaws distal ends 311 and gradually progressing towards forceps jaws proximal ends 312. In one or more embodiments, a compression of handle 110 may be configured to close a first forceps jaw 310 and a second forceps jaw 310 wherein the first and second forceps jaws 310 initially contact each other at first and second forceps jaws distal ends 311. Illustratively, a compression of handle 110 may be configured to close a first membrane hook 340 and a second membrane hook 340 wherein the first and second membrane hooks 340 initially contact each other at first and second membrane hook distal ends 341. In one or more embodiments, after the first and second forceps jaws 310 initially contact at first and second forceps jaws distal ends 311, a compression of handle 110 may be configured to cause medial portions of the first and second forceps jaws 310 to gradually contact each other starting at medial portions of the first and second forceps jaws 310 adjacent to first and second forceps jaws distal ends 311.

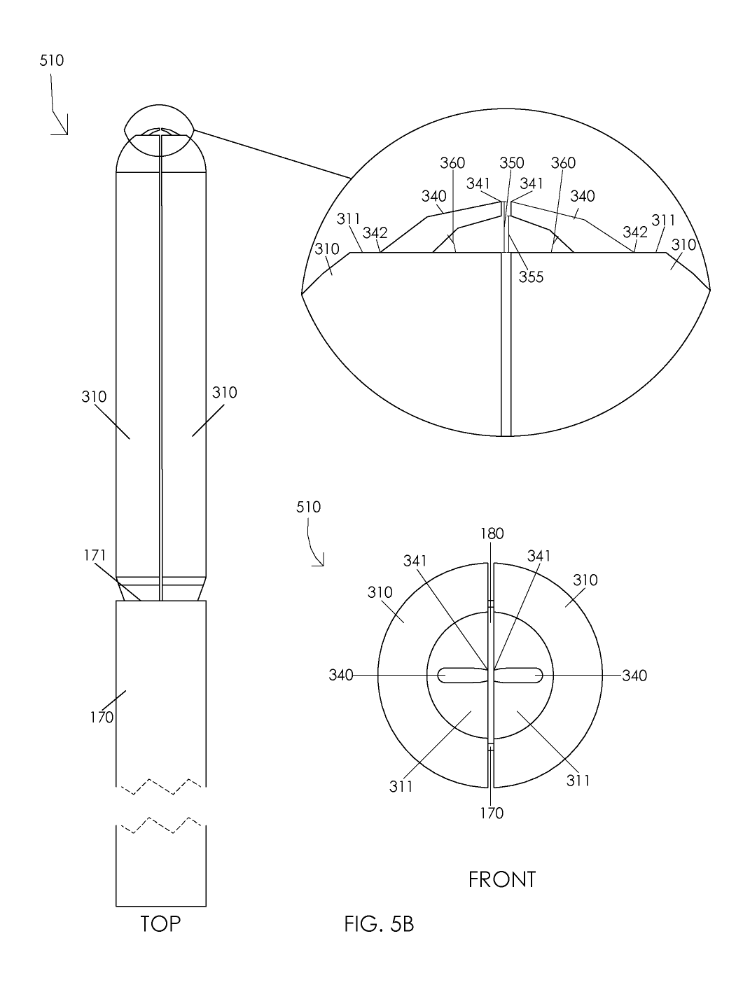

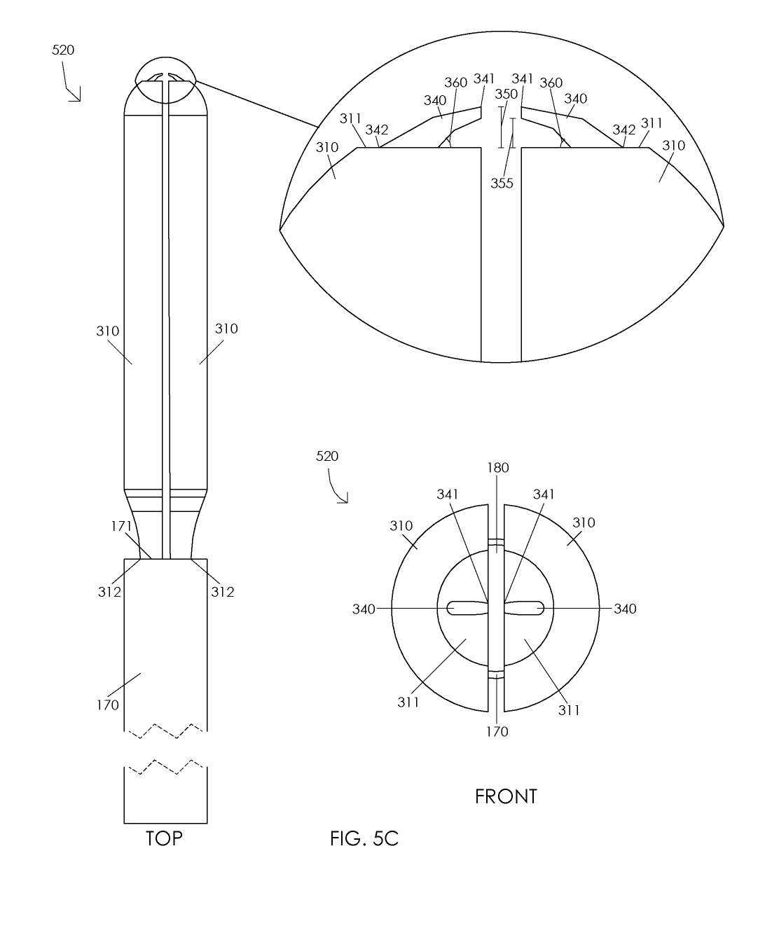

FIGS. 5A, 5B, and 5C are schematic diagrams illustrating a gradual opening of a membrane removing forceps 300. FIG. 5A illustrates a top view and a front view of a closed membrane removing forceps 500. In one or more embodiments, membrane removing forceps 300 may comprise a closed membrane removing forceps 500, e.g., when a first forceps jaw distal end 311 is adjacent to a second forceps jaw distal end 311. Illustratively, membrane removing forceps 300 may comprise a closed membrane removing forceps 500, e.g., when outer hypodermic tube 170 is fully extended over forceps jaws proximal ends 312. In one or more embodiments, membrane removing forceps 300 may comprise a closed membrane removing forceps 500, e.g., when handle 110 is fully compressed.

FIG. 5B illustrates a top view and a front view of a partially open membrane removing forceps 510. In one or more embodiments, a decompression of handle 110 may be configured to gradually open a membrane removing forceps 300, e.g., from a closed membrane removing forceps 500 to a partially open membrane removing forceps 510. Illustratively, a decompression of handle 110 may be configured to retract outer hypodermic tube 170 relative to surgical blank 180, e.g., a decompression of handle 110 may be configured to retract outer hypodermic tube distal end 171 relative to forceps jaws proximal ends 312. In one or more embodiments, a decompression of handle 110 may be configured to gradually separate forceps jaws 310. Illustratively, a decompression of handle 110 may be configured to gradually separate forceps jaws 310 wherein a first forceps jaw distal end 311 contacts a second forceps jaw distal end 311 until all other portions of forceps jaws 310 are separated. In one or more embodiments, a decompression of handle 110 may be configured to separate forceps jaws 310 wherein forceps jaws distal ends 311 are the last portions of forceps jaws 310 to separate.

FIG. 5C illustrates a top view and a front view of a fully open membrane removing forceps 520. Illustratively, a decompression of handle 110 may be configured to gradually open a membrane removing forceps 300, e.g., from a partially open membrane removing forceps 510 to a fully open membrane removing forceps 520. In one or more embodiments, a decompression of handle 110 may be configured to retract outer hypodermic tube 170 relative to surgical blank 180, e.g., a decompression of handle 110 may be configured to retract outer hypodermic tube distal end 171 relative to forceps jaws proximal ends 312. Illustratively, a decompression of handle 110 may be configured to gradually separate forceps jaws 310. In one or more embodiments, a first forceps jaw distal end 311 and a second forceps jaw distal end 311 may be separated by forceps jaw maximum separation distance 315, e.g., when membrane removing forceps 300 comprises a fully open membrane removing forceps 520.

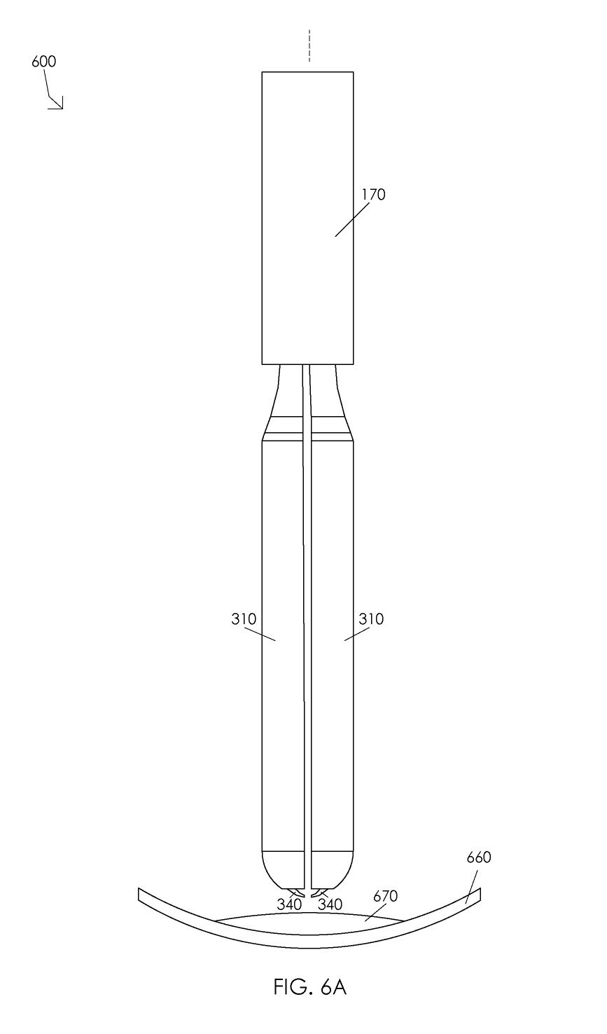

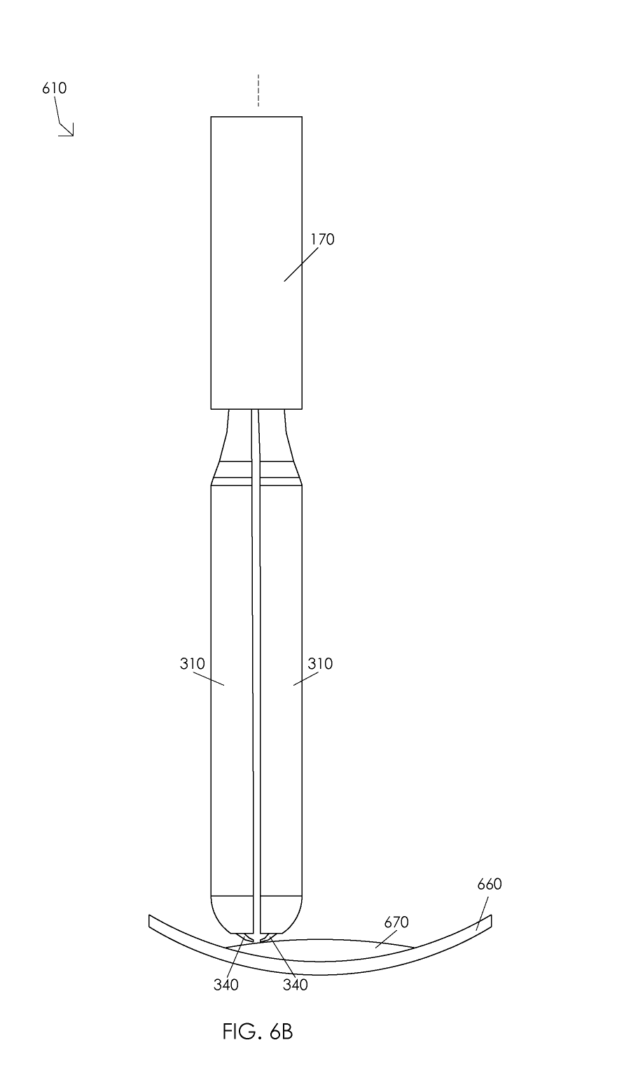

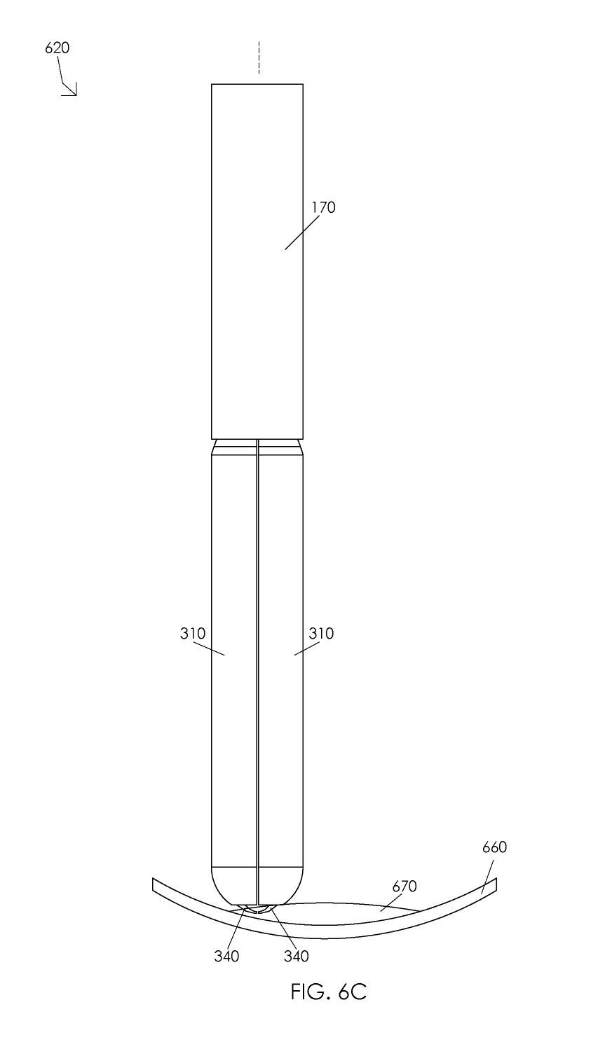

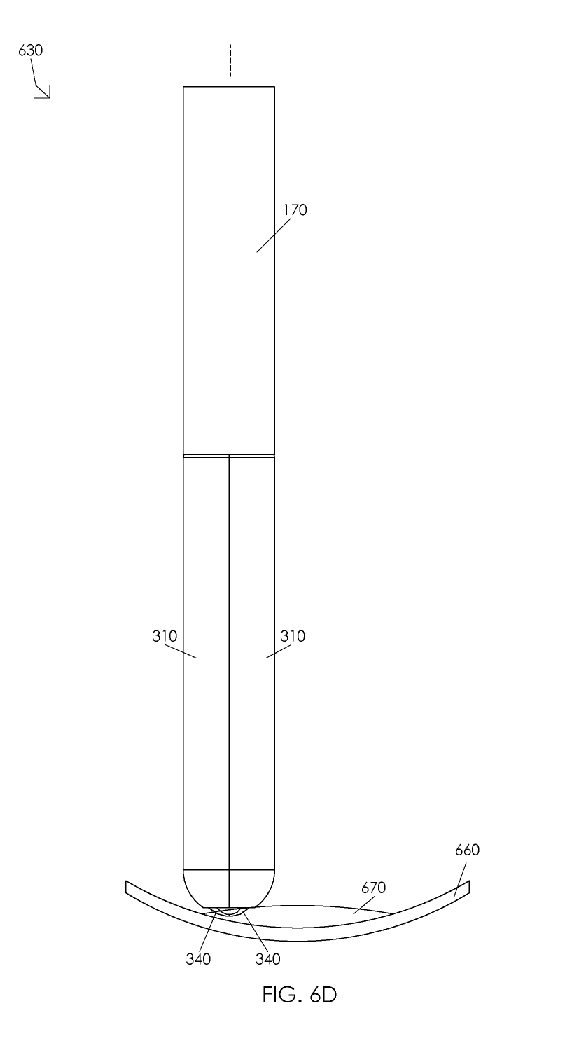

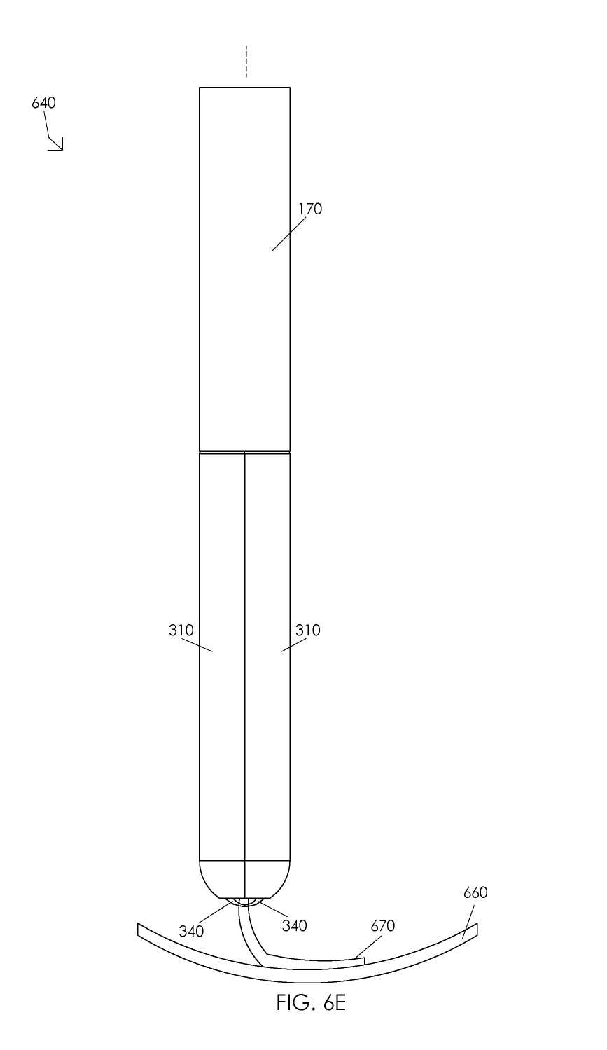

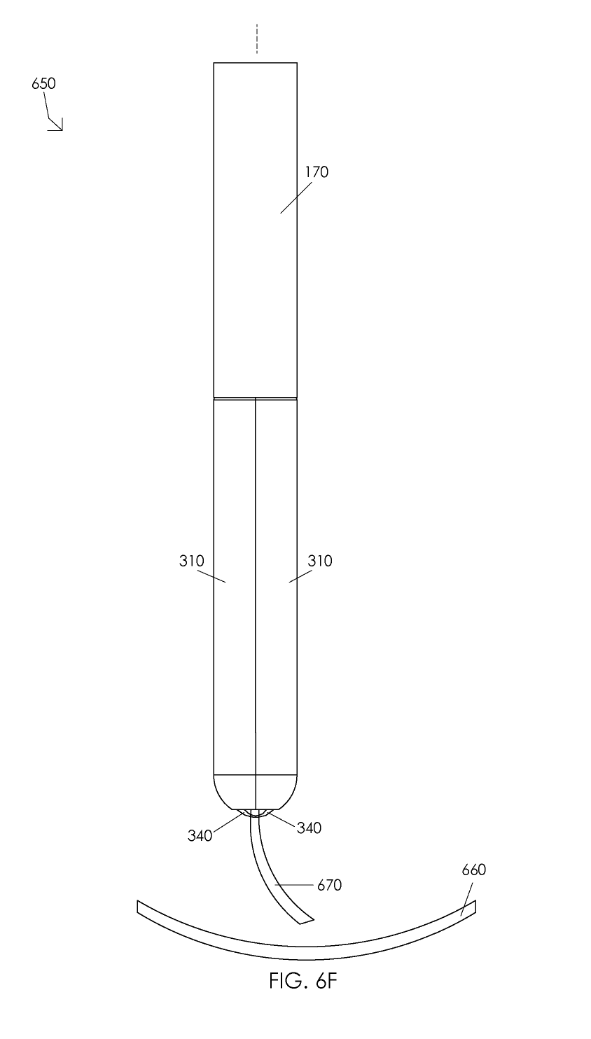

FIGS. 6A, 6B, 6C, 6D, 6E and 6F are schematic diagrams illustrating a membrane removal. FIG. 6A illustrates an attached membrane 600. Illustratively, an attached membrane 600 may comprise an internal limiting membrane 670 attached to a retina 660. In one or more embodiments, a surgeon may remove an attached membrane 600 by grasping a portion of a membrane between forceps jaws 310 and peeling the membrane off of an underlying tissue. Illustratively, a surgeon may remove an internal limiting membrane 670 attached to a retina 660 by grasping a portion of internal limiting membrane 670 between forceps jaws 310 and peeling internal limiting membrane 670 off of retina 660. In one or more embodiments, a surgeon may remove an attached membrane 600 by grasping a portion of a membrane between membrane hooks 340 and peeling the membrane off of an underlying tissue. Illustratively, a surgeon may remove an internal limiting membrane 670 attached to a retina 660 by grasping a portion of internal limiting membrane 670 between membrane hooks 340 and peeling internal limiting membrane 670 off of retina 660.

FIG. 6B illustrates an initial membrane contact 610. Illustratively, a surgeon may manipulate membrane removing forceps 300 within an eye to cause a contact between a portion of membrane removing forceps 300 and a portion of an attached membrane 600, e.g., a surgeon may manipulate membrane removing forceps 300 within an eye to cause an initial membrane contract 610. In one or more embodiments, an initial membrane contact 610 may comprise a contact between a portion of membrane hook 340 and a portion of a membrane, e.g., an initial membrane contact 610 may comprise a contact between membrane hook distal end 341 and internal limiting membrane 670.

FIG. 6C illustrates a membrane piercing 620. Illustratively, a surgeon may manipulate membrane removing forceps 300 within an eye to cause a portion of membrane removing forceps 300 to penetrate into a membrane, e.g., a surgeon may manipulate membrane removing forceps 300 within an eye to penetrate membrane hooks 340 into internal limiting membrane 670. In one or more embodiments, a surgeon may manipulate membrane removing forceps 300 within an eye to cause a portion of membrane removing forceps 300 to penetrate into a membrane without damaging a tissue underlying the membrane, e.g., a surgeon may manipulate membrane removing forceps 300 within an eye to cause membrane hooks 340 to penetrate into internal limiting membrane 670 without damaging retina 660. Illustratively, a surgeon may manipulate membrane removing forceps 300 within an eye to cause a membrane piercing 620. In one or more embodiments, a membrane piercing 620 may comprise penetrating a portion of membrane removing forceps 300 into a membrane, e.g., a membrane piercing 620 may comprise penetrating membrane hooks 340 in to internal limiting membrane 670. Illustratively, a membrane piercing 620 may be configured to prevent damage to a tissue underlying a pierced membrane, e.g., one or more properties of membrane removing forceps 300 may be configured to prevent damage to a tissue underlying a pierced membrane. In one or more embodiments, membrane hook outer height 350 may be configured to prevent damage to a tissue underlying a membrane, e.g., membrane hook outer height 350 may be a distance that is less than the average thickness of the membrane. Illustratively, a surface area of forceps jaw distal end 311 may be configured to prevent damage to a tissue underlying a membrane. In one or more embodiments, membrane removing forceps 300 may be configured to perform a membrane piercing 620 wherein only membrane hooks 340 penetrate a membrane, e.g., membrane removing forceps 300 may be configured to perform a membrane piercing 620 wherein only membrane hooks 340 penetrate internal limiting membrane 670. Illustratively, a surface area of forceps jaws distal ends 311 may be configured to prevent any portion of membrane removing forceps 300 other than membrane hooks 340 from penetrating a membrane. In one or more embodiments, a ratio of a surface area of each forceps jaw distal end 311 to a surface area of each membrane hook 340 may be in a range of 750.0 to 1500.0, e.g., a ratio of a surface area of each forceps jaw distal end 311 to a surface area of each membrane hook 340 may be 1334.7. Illustratively, a ratio of a surface area of each forceps jaw distal end 311 to a surface area of each membrane hook 340 may be less than 750.0 or greater than 1500.0. In one or more embodiments, a ratio of a surface area of each forceps jaw distal end 311 to a surface area of each membrane hook 340 may large enough to allow membrane hook 340 to penetrate a membrane and prevent forceps jaw distal end 311 from penetrating the membrane. Illustratively, a ratio of a surface area of each forceps jaw distal end 311 to a surface area of each membrane hook 340 may large enough to allow membrane hook 340 to penetrate internal limiting membrane 670 and prevent forceps jaw distal end 311 from penetrating internal limiting membrane 670.

FIG. 6D illustrates a membrane grasping 630. Illustratively, a membrane grasping 630 may comprise disposing a portion of a membrane between forceps jaws 310, e.g., a membrane grasping 630 may comprise disposing a portion of a membrane between forceps jaws distal ends 311. In one or more embodiments, a membrane gasping 630 may comprise disposing a portion of internal limiting membrane 670 between forceps jaws distal ends 311. Illustratively, a membrane grasping 630 may comprise disposing a portion of a membrane between membrane hooks 340, e.g., a membrane grasping 630 may comprise disposing a portion of a membrane between membrane hooks distal ends 341. In one or more embodiments, a membrane grasping 630 may comprise disposing a portion of internal limiting membrane 670 between membrane hooks distal ends 341. Illustratively, a surgeon may perform a membrane grasping 630 by compressing handle 110, e.g., a surgeon may perform a membrane grasping 630 by compressing handle 110 after a membrane piercing 620. In one or more embodiments, a compression of handle 110 may be configured to extend outer hypodermic tube 170 relative to surgical blank 180, e.g., a compression of handle 110 may be configured to extend outer hypodermic tube distal end 171 over forceps jaws proximal ends 312. Illustratively, a compression of handle 110 may be configured to decrease a distance between a first membrane hook distal end 341 and a second membrane hook distal end 341. In one or more embodiments, decreasing a distance between a first membrane hook distal end 341 and a second membrane hook distal end 341 may be configured to perform a membrane grasping 630.

FIG. 6E illustrates a partially removed membrane 640. Illustratively, a partially removed membrane 640 may comprise a membrane partially separated from an underlying tissue, e.g., a partially removed membrane 640 may comprise an internal limiting membrane 670 partially separated from a retina 660. In one or more embodiments, a surgeon may peel a membrane apart from an underlying tissue by grasping the membrane with membrane removing forceps 300 and pulling the membrane apart from the underlying tissue, e.g., a surgeon may peel a membrane apart from an underlying tissue by performing a membrane grasping 630 and pulling the membrane apart from the underlying tissue. Illustratively, a surgeon may peel a membrane apart from an underlying tissue by grasping the membrane with membrane removing forceps 300 and pulling the membrane apart from the underlying tissue until the membrane comprises a partially removed membrave 640. In one or more embodiments, a surgeon may peel a membrane apart from an underlying tissue by grasping the membrane with membrane hooks 340 and pulling the membrane apart from the underlying tissue until the membrane comprises a partially removed membrane 640. Illustratively, a surgeon may peel internal limiting membrane 670 apart from an underlying retina 660 by grasping internal limiting membrane 670 with membrane removing forceps 300 and pulling internal limiting membrane 670 apart from retina 660 until internal limiting membrane 670 comprises a partially removed membrane 640. In one or more embodiments, a surgeon may peel internal limiting membrane 670 apart from an underlying retina 660 by grasping internal limiting membrane 670 with membrane hooks 340 and pulling internal limiting membrane 670 apart from retina 660 until internal limiting membrane 670 comprises a partially removed membrane 640.

FIG. 6F illustrates a fully removed membrane 650. Illustratively, a fully removed membrane 650 may comprise a membrane completely separated from an underlying tissue, e.g., a fully removed membrane 650 may comprise an internal limiting membrane 670 completely separated from retina 660. In one or more embodiments, a surgeon may peel a membrane apart from an underlying tissue by grasping the membrane with membrave removing forceps 300 and pulling the membrane apart from the underlying tissue until the membrane comprises a fully removed membrane 650. Illustratively, a surgeon may peel a membrane apart from an underlying tissue by grasping the membrane with membrane hooks 340 and pulling the membrane apart from the underlying tissue until the membrane comprises a fully removed membrane 650. In one or more embodiments, a surgeon may peel a membrane apart from an underlying tissue by grasping the membrane with membrane hooks 340 and pulling the membrane apart from the underlying tissue until the membrane comprises a partially removed membrane 640, e.g., a surgeon may then the grasp the membrane with forceps jaws 310 and peel the membrane apart from the underlying tissue until the membrane comprises a fully removed membrane 650. Illustratively, a surgeon may continue to peel a partially removed membrane 640 apart from an underlying tissue until the membrane comprises a fully removed membrane 650. In one or more embodiments, a surgeon may peel internal limiting membrane 670 apart from retina 660 by grasping internal limiting membrane 670 with membrane removing forceps 300 and pulling internal limiting membrane 670 apart from retina 660 until internal limiting membrane 670 comprises a fully removed membrane 650. Illustratively, a surgeon may peel internal limiting membrane 670 apart from retina 660 by grasping internal limiting membrane 670 with membrane hooks 340 and pulling internal limiting membrane 670 apart from retina 660 until internal limiting membrane 670 comprises a fully removed membrane 650. In one or more embodiments, a surgeon may peel internal limiting membrane 670 apart from retina 660 by grasping internal limiting membrane 670 with membrane hooks 340 and pulling internal limiting membrane 670 apart from retina 660 until internal limiting membrane 670 comprises a partially removed membrane 640, e.g., a surgeon may then the grasp internal limiting membrane 670 with forceps jaws 310 and peel internal limiting membrane 670 apart from retina 660 until internal limiting membrane 670 comprises a fully removed membrane 650. Illustratively, a surgeon may continue to peel a partially removed membrane 640 apart from retina 660 until internal limiting membrane 670 comprises a fully removed membrane 650.

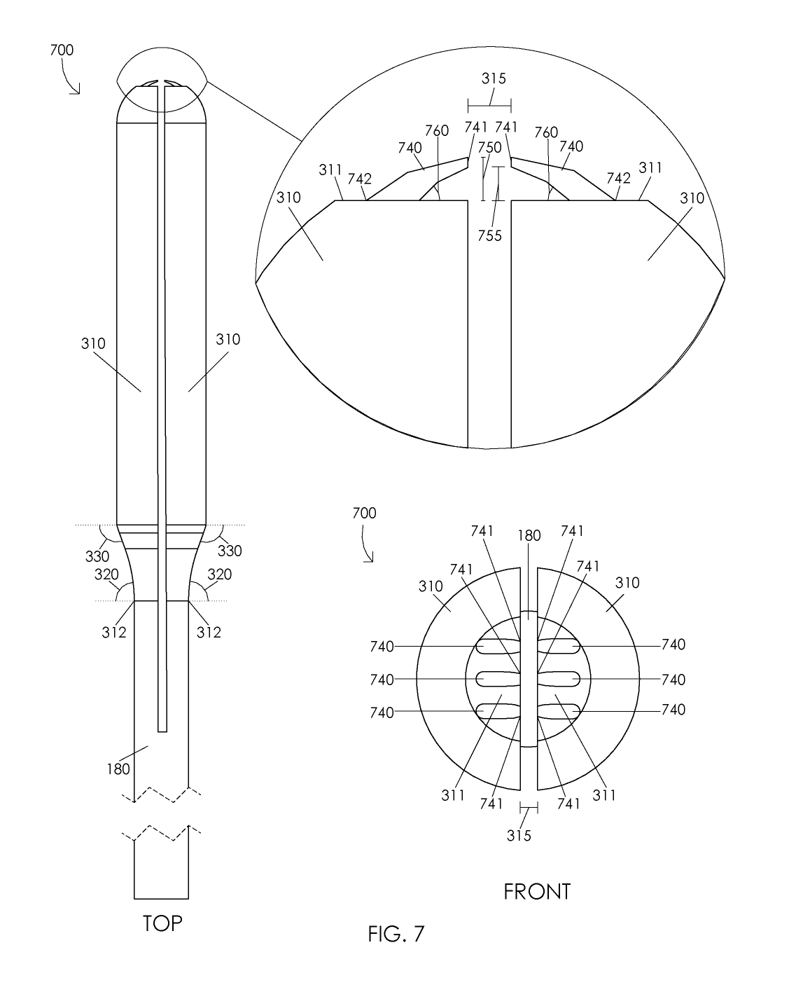

FIG. 7 is a schematic diagram illustrating a membrane removing forceps 700. FIG. 7 illustrates a top view and a front view of a membrane removing forceps 700. Illustratively, membrane removing forceps 700 may be manufactured with dimensions configured for performing microsurgical procedures, e.g., ophthalmic surgical procedures. In one or more embodiments, membrane removing forceps 700 may be manufactured from surgical blank 180. In one or more embodiments, membrane removing forceps 700 may be manufactured by modifying surgical blank 180, e.g., with an electric discharge machine, a laser, a file, deep reactive ion etching, or any suitable modification means. Illustratively, membrane removing forceps 700 may comprise a plurality of forceps jaws 310 wherein each forceps jaw 310 has a forceps jaw distal end 311 and a forceps jaw proximal end 312. In one or more embodiments, each forceps jaw distal end 311 may have a surface area in a range of 0.03 to 0.15 square millimeters, e.g., each forceps jaw distal end 311 may have a surface area of 0.065 square millimeters. Illustratively, each forceps jaw distal end 311 may have a surface area less than 0.03 square millimeters or greater than 0.15 square millimeters. In one or more embodiments, forceps jaws distal ends 311 may be separated by a forceps jaw maximum separation distance 315. Illustratively, forceps jaw maximum separation distance 315 may comprise a distance in a range of 50.0 to 800.0 micrometers, e.g., forceps jaw maximum separation distance 315 may comprise a distance of 600 micrometers. In one or more embodiments, a geometry of membrane removing forceps 700 may comprise a first contour angle 320 and a second contour angle 330. Illustratively, first contour angle 320 may comprise any angle less than or equal to 90.0 degrees, e.g., first contour angle 320 may comprise an 80.0 degree angle. In one or more embodiments, first contour angle 320 may comprise an angle in a range of 60.0 to 80.0 degrees, e.g., first contour angle 320 may comprise a 72.3 degree angle. Illustratively, first contour angle 320 may comprise an angle less than 60.0 degrees or greater than 80.0 degrees. In one or more embodiments, second contour angle 330 may comprise any angle greater than or equal to 90.0 degrees, e.g., second contour angle 330 may comprise a 100.0 degree angle. Illustratively, second contour angle 330 may comprise an angle in a range of 95.0 to 120.0 degrees, e.g., second contour angle 330 may comprise a 107.0 degree angle. In one or more embodiments, second contour angle 330 may comprise an angle less than 95.0 degrees or greater than 120.0 degrees.

Illustratively, membrane removing forceps 700 may comprise a plurality of membrane hooks 740 wherein each membrane hook 740 has a membrane hook distal end 741 and a membrane hook proximal end 742. In one or more embodiments, each forceps jaw 310 may comprise a plurality of membrane hooks 740. Illustratively, each membrane hook 740 extends from a forceps jaw distal end 311, e.g., forceps jaw distal end 311 may be adjacent to membrane hook proximal end 742. In one or more embodiments, a first membrane hook 740 may extend from a first forceps jaw distal end 311 and a second membrane hook 740 may extend from a second forceps jaw distal end 311. Illustratively, a first membrane hook 740 and a second membrane hook 740 may extend from a first forceps jaw distal end 311 and a third membrane hook 740 may extend from a second forceps jaw distal end 311. In one or more embodiments, a first membrane hook 740 and a second membrane hook 740 may extend from a first forceps jaw distal end 311 and a third membrane hook 740 and a fourth membrane hook 740 may extend from a second forceps jaw distal end 311. Illustratively, a first membrane hook 740, a second membrane hook 740, and a third membrane hook 740 may extend from a first forceps jaw distal end 311 and a fourth membrane hook 740 may extend from a second forceps jaw distal end 311. In one or more embodiments, a first membrane hook 740, a second membrane hook 740, and a third membrane hook 740 may extend from a first forceps jaw distal end 311 and a fourth membrane hook 740 and a fifth membrane hook 740 may extend from a second forceps jaw distal end 311. Illustratively, a first membrane hook 740, a second membrane hook 740, and a third membrane hook 740 may extend from a first forceps jaw distal end 311 and a fourth membrane hook 740, a fifth membrane hook 740, and a sixth membrane hook 740 may extend from a second forceps jaw distal end 311. Illustratively, membrane hook 740 may be configured to grasp a portion of a membrane, e.g., membrane hook 740 may be configured to grasp a portion of an internal limiting membrane 670. In one or more embodiments, membrane hook 740 may be configured to grasp a portion of a first tissue disposed over a second tissue without damaging the second tissue. Illustratively, membrane hook 740 may be configured to grasp a first tissue having a convex surface geometry disposed over a second tissue having a convex surface geometry without damaging the second tissue. In one or more embodiments, each membrane hook 740 may have a surface area in a range of 25.0 to 75.0 square micrometers, e.g., each membrane hook 740 may have a surface area of 48.7 square micrometers. Illustratively, each membrane hook 740 may have a surface area less than 25.0 square micrometers or greater than 75.0 square micrometers.

Illustratively, each membrane hook 740 may comprise a membrane hook outer height 750, a membrane hook inner height 755, and a membrane hook angle 760. In one or more embodiments, membrane hook angle 760 may comprise any angle less than 90.0 degrees, e.g., membrane hook angle 760 may comprise a 45.0 degree angle. Illustratively, membrane hook angle 760 may comprise an angle in a range of 5.0 to 20.0 degrees, e.g., membrane hook angle 760 may comprise a 10.0 degree angle. In one or more embodiments, membrane hook angle 760 may comprise an angle less than 5.0 degrees or greater than 20.0 degrees, e.g., membrane hook angle 760 may comprise a 3.8 degree angle. Illustratively, membrane hook outer height 750 may be configured to prevent damage to a tissue underlying a membrane, e.g., membrane hook outer height 750 may be configured to prevent damage to a retina 660 underlying an internal limiting membrane 670. In one or more embodiments, membrane hook outer height 750 may comprise a distance that is a fraction of an average membrane thickness, e.g., membrane hook outer height 750 may comprise a distance that is a fraction of an average internal limiting membrane thickness. Illustratively, membrane hook outer height 750 may comprise a distance in a range of 0.25 to 3.0 micrometers, e.g., membrane hook outer height 750 may comprise a distance of 1.25 micrometers. In one or more embodiments, membrane hook outer height 750 may comprise a distance less than 0.25 micrometers or greater than 3.0 micrometers, e.g., membrane hook outer height 750 may comprise a distance of 0.15 micrometers. Illustratively, a particular membrane hook outer height 750 may be selected, e.g., by a surgeon, on a case-by-case basis. For example, a surgeon may select a first membrane removing forceps 700 having a first membrane hook outer height 750 to remove a first membrane and the surgeon may select a second membrane removing forceps 700 having a second membrane hook outer height 750 to remove a second membrane wherein the first membrane is thicker than the second membrane and the first membrane hook outer height 750 is greater than the second membrane hook outer height 750. Illustratively, membrane hook inner height 755 may be configured to grasp a portion of a membrane, e.g., membrane hook inner height 755 may be configured to grasp a portion of an internal limiting membrane 670. In one or more embodiments, membrane hook inner height 755 may comprise any distance less than membrane hook outer height 750, e.g., membrane hook inner height 755 may comprise a distance equal to 80.0 percent of membrane hook outer height 750. Illustratively, membrane hook inner height 755 may comprise a distance in a range of 0.1 to 2.9 micrometers, e.g., membrane hook inner height 755 may comprise a distance of 1.0 micrometers. In one or more embodiments, membrane hook inner height 755 may comprise a distance less than 0.1 micrometers or greater than 2.9 micrometers, e.g., membrane hook inner height 755 may comprise a distance of 0.05 micrometers.

Illustratively, membrane hook 740 may be manufactured by modifying surgical blank 180, e.g., by laser ablation. In one or more embodiments, membrane hook 740 may be manufactured by modifying surgical blank 180, e.g., by femtosecond laser ablation. Illustratively, membrane hook 740 may be manufactured by laser ablation of surgical blank 180 in multiple orientations. In one or more embodiments, membrane hook 740 may be manufactured by performing laser ablation of surgical blank 180 in a first orientation, e.g., and then performing laser ablation of surgical blank 180 in a second orientation. Illustratively, membrane hook 740 may be manufactured by performing a first laser ablation of surgical blank 180 and then rotating surgical blank 180 to perform a second laser ablation of surgical blank 180. In one or more embodiments, membrane hook 740 may be manufactured by performing a first laser ablation of surgical blank 180 and then rotating surgical blank 180 by 90.0 degrees to perform a second laser ablation of surgical blank 180. Illustratively, membrane hook 740 may be formed by high precision micromachining using a 355 nm Nd: vanadate laser operating at 10 kHz and with an average power of 7.0 Watts and pulse duration of 35.0 nanoseconds. In one or more embodiments, membrane hook 740 may be manufactured by using an electric discharge machine to shape membrane removing forceps 700 from blank 180 and then using laser micromachining to shape membrane hook 740. Illustratively, membrane hook 740 may be manufactured by modifying surgical blank 180, e.g., by deep reactive-ion etching. In one or more embodiments, membrane hook 740 may be manufactured by modifying surgical blank 180, e.g., by the Bosch process of time-multiplexed etching. Illustratively, membrane hook 740 may be manufactured by exposing a portion of surgical blank 180 to repeated cycles of isotropic plasma etching followed by deposition of a chemically inert passivation layer. In one or more embodiments, membrane hook 740 may be manufactured by fabricating a membrane hook 740 on a substrate and then fixing the substrate to a portion of surgical blank 180. Illustratively, surgical blank 180 may be modified, e.g., by deep reactive-ion etching, to manufacture membrane hook 740. In one or more embodiments, surgical blank 180 may be modified wherein one or more portions of surgical blank 180 comprise membrane hook 740 and then surgical blank 180 may be modified to manufacture membrane removing forceps 700. Illustratively, membrane hook 740 may be manufactured by deep reactive-ion etching of surgical blank 180 in multiple orientations. In one or more embodiments, membrane hook 740 may be manufactured by performing deep reactive-ion etching of surgical blank 180 in a first orientation, e.g., and then performing deep reactive-ion etching of surgical blank 180 in a second orientation. Illustratively, membrane hook 740 may be manufactured by performing a first deep reactive-ion etching of surgical blank 180 and then rotating surgical blank 180 to perform a second deep reactive-ion etching of surgical blank 180. In one or more embodiments, membrane hook 740 may be manufactured by performing a first deep reactive-ion etching of surgical blank 180 and then rotating surgical blank 180 by 90.0 degrees to perform a second deep reactive-ion etching of surgical blank 180. Illustratively, surgical blank 180 may be modified, e.g., by an electric discharge machine, to manufacture membrane removing forceps 700 and then membrane removing forceps 700 may be modified, e.g., by deep reactive-ion etching, to fabricate membrane hook 740.

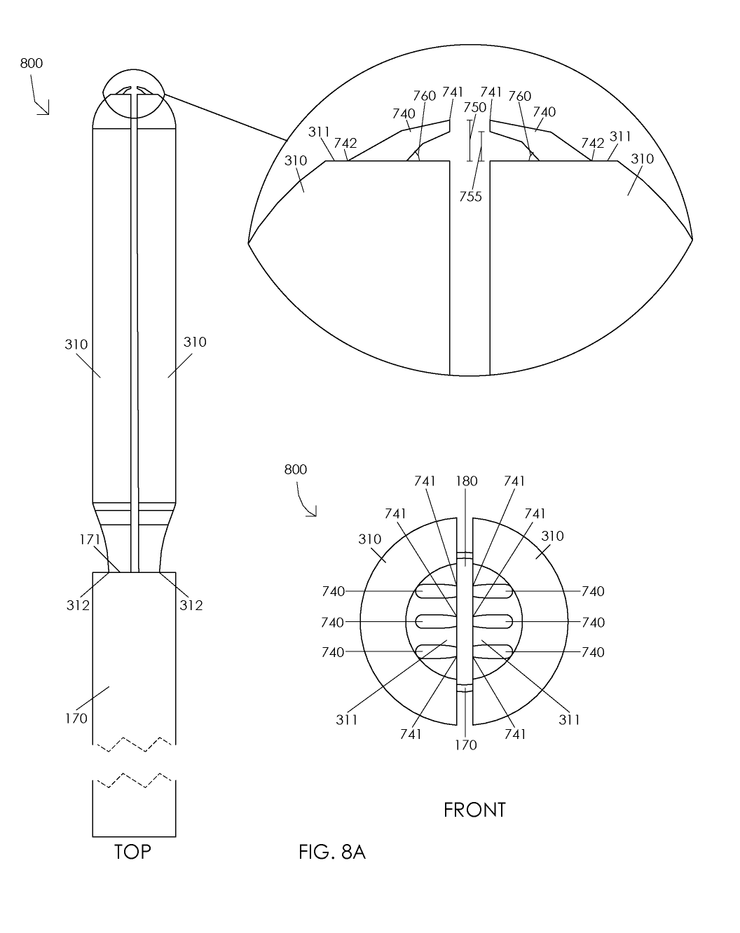

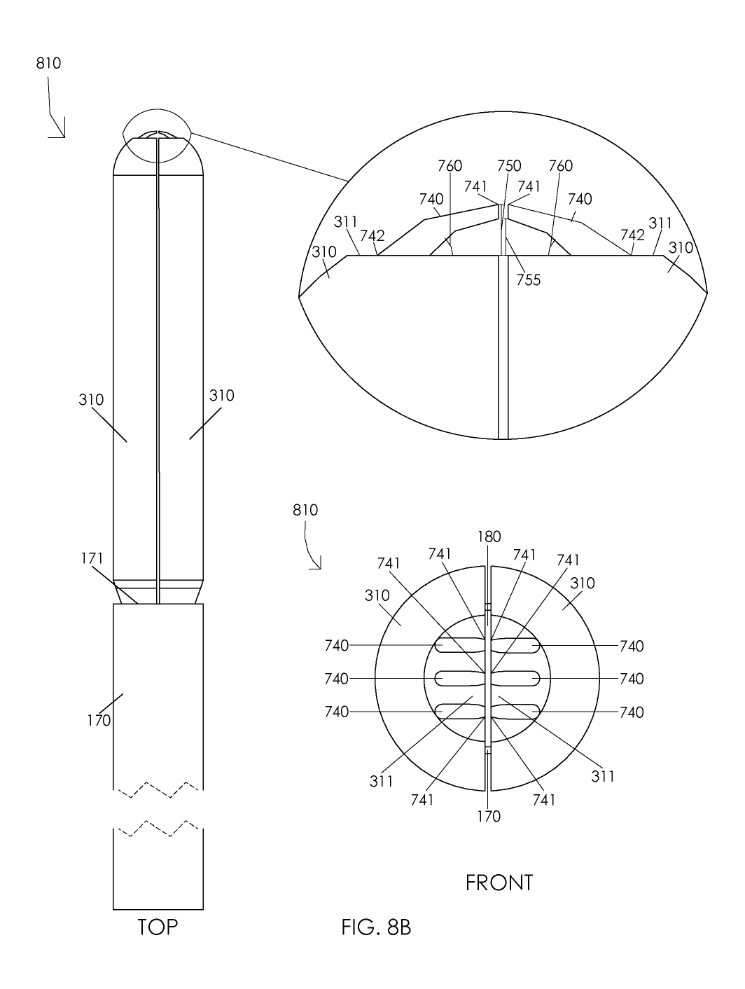

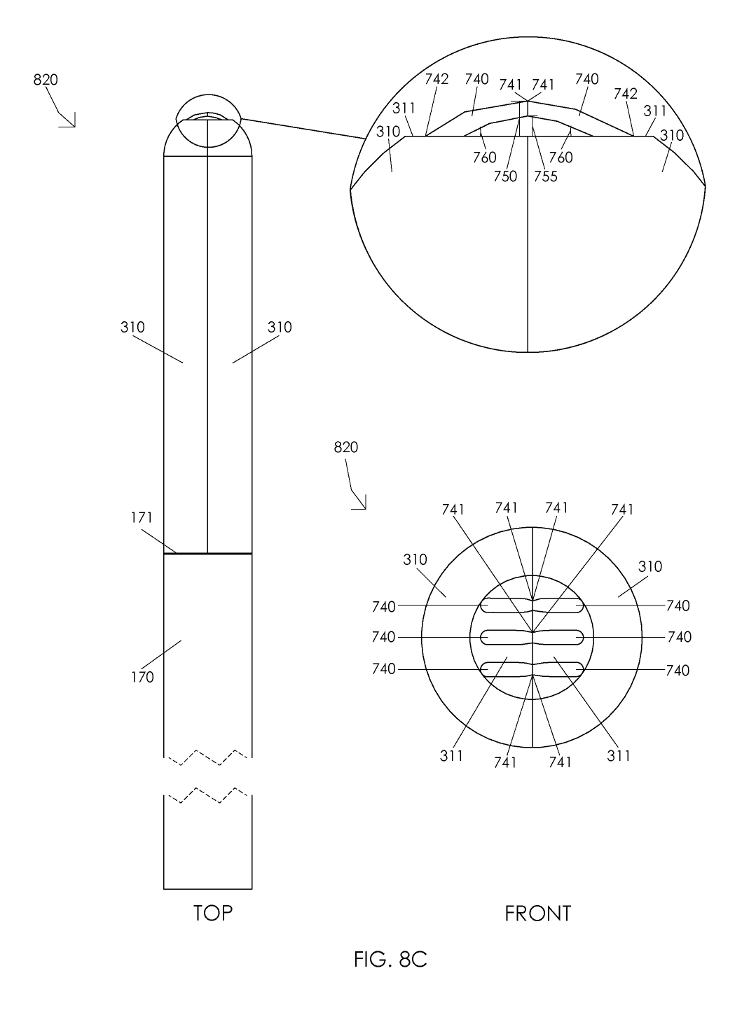

FIGS. 8A, 8B, and 8C are schematic diagrams illustrating a gradual closing of a membrane removing forceps 700. FIG. 8A illustrates a top view and a front view of an open membrane removing forceps 800. In one or more embodiments, membrane removing forceps 700 may comprise an open membrane removing forceps 800, e.g., when a first forceps jaw distal end 311 is separated from a second forceps jaw distal end 311 by forceps jaw maximum separation distance 315. Illustratively, membrane removing forceps 700 may comprise an open membrane removing forceps 800, e.g., when outer hypodermic tube 170 is fully retracted relative to forceps jaws proximal ends 312. Illustratively, membrane removing forceps 700 may comprise an open membrane removing forceps 800, e.g., when handle 110 is fully decompressed.

FIG. 8B illustrates a top view and a front view of a partially closed membrane removing forceps 810. In one or more embodiments, a compression of handle 110 may be configured to gradually close a membrane removing forceps 700, e.g., from an open membrane removing forceps 800 to a partially closed membrane removing forceps 810. Illustratively, a compression of handle 110 may be configured to extend outer hypodermic tube 170 relative to surgical blank 180, e.g., a compression of handle 110 may be configured to extend outer hypodermic tube distal end 171 over forceps jaws proximal ends 312. In one or more embodiments, a compression of handle 110 may be configured to decrease a distance between a first forceps jaw distal end 311 and a second forceps jaw distal end 311, e.g., a first forceps jaw distal end 311 and a second forceps jaw distal end 311 may be separated by a distance less than forceps jaw maximum separation distance 315 when membrane removing forceps 700 comprises a partially closed membrane removing forceps 810.

FIG. 8C illustrates a top view and a front view of a fully closed membrane removing forceps 820. Illustratively, a compression of handle 110 may be configured to gradually close a membrane removing forceps 700, e.g., from a partially closed membrane removing forceps 810 to a fully closed membrane removing forceps 820. In one or more embodiments, a compression of handle 110 may be configured to extend outer hypodermic tube 170 relative to surgical blank 180, e.g., a compression of handle 110 may be configured to extend outer hypodermic tube distal end 171 over forceps jaws proximal ends 312. Illustratively, an extension of outer hypodermic tube 170 over forceps jaws proximal ends 312 may be configured to close forceps jaws 310 wherein forceps jaws 310 initially contact at forceps jaws distal ends 311. In one or more embodiments, an extension of outer hypodermic tube 170 over forceps jaws proximal end 312 may be configured to close forceps jaws 310 wherein membrane hooks 740 initially contact at membrane hooks distal ends 741. Illustratively, a compression of handle 110 may be configured to gradually close forceps jaws 310 wherein forceps jaws 310 initially contact at forceps jaws distal ends 311. In one or more embodiments, a compression of handle 110 may be configured to gradually close forceps jaws 310 wherein membrane hooks 740 initially contact at membrane hooks distal ends 741. Illustratively, after forceps jaws distal ends 311 initially contact, a compression of handle 110 may be configured to gradually close forceps jaws 310 wherein a contact area between forceps jaws 310 gradually increases. In one or more embodiments, forceps jaws 310 may be configured to close wherein an amount of a first forceps jaw 310 in contact with a second forceps jaw 310 increases gradually from forceps jaws distal ends 311, e.g., forceps jaws 310 may be configured to close wherein an amount of a first forceps jaw 310 in contact with a second forceps jaw 310 increases gradually towards forceps jaws proximal ends 312. Illustratively, a compression of handle 110 may be configured to close forceps jaws 310 starting at forceps jaws distal ends 311 and gradually progressing towards forceps jaws proximal ends 312. In one or more embodiments, a compression of handle 110 may be configured to close a first forceps jaw 310 and a second forceps jaw 310 wherein the first and second forceps jaws 310 initially contact each other at first and second forceps jaws distal ends 311. Illustratively, a compression of handle 110 may be configured to close a first membrane hook 740 and a second membrane hook 740 wherein the first and second membrane hooks 740 initially contact each other at first and second membrane hook distal ends 741. In one or more embodiments, after the first and second forceps jaws 310 initially contact at first and second forceps jaws distal ends 311, a compression of handle 110 may be configured to cause medial portions of the first and second forceps jaws 310 to gradually contact each other starting at medial portions of the first and second forceps jaws 310 adjacent to first and second forceps jaws distal ends 311.

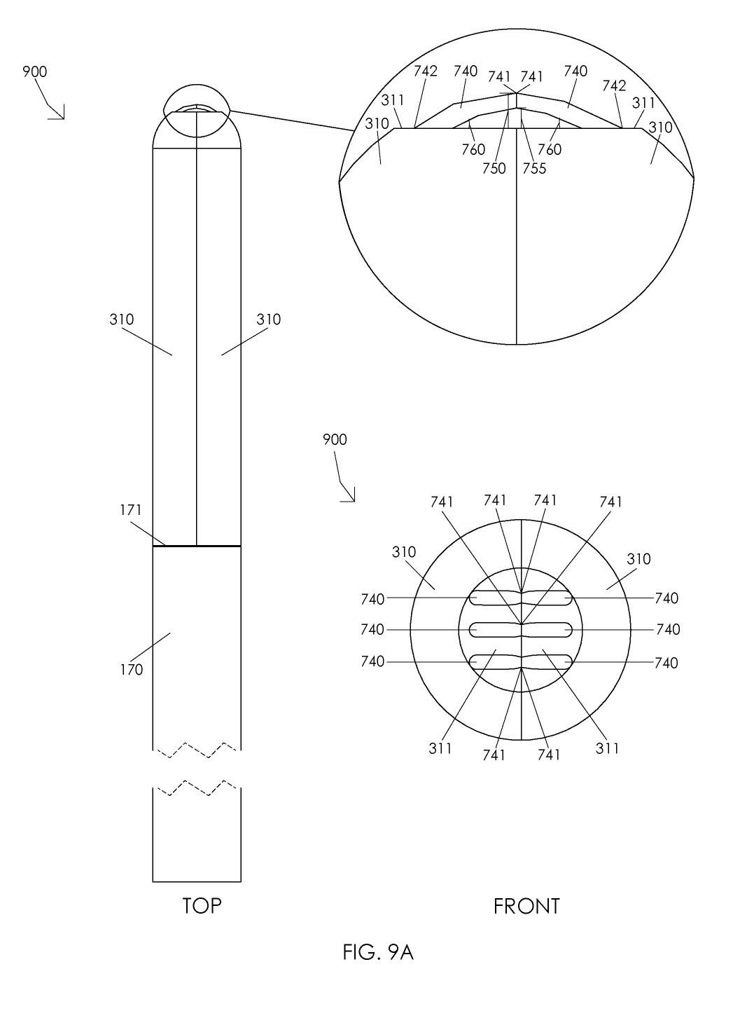

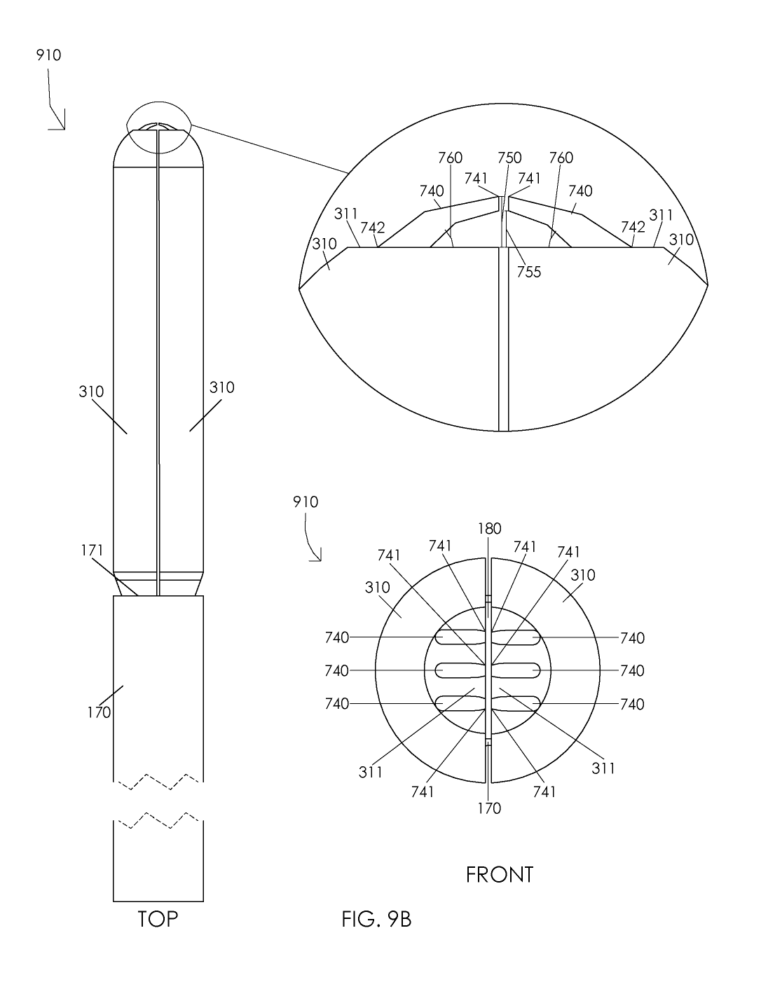

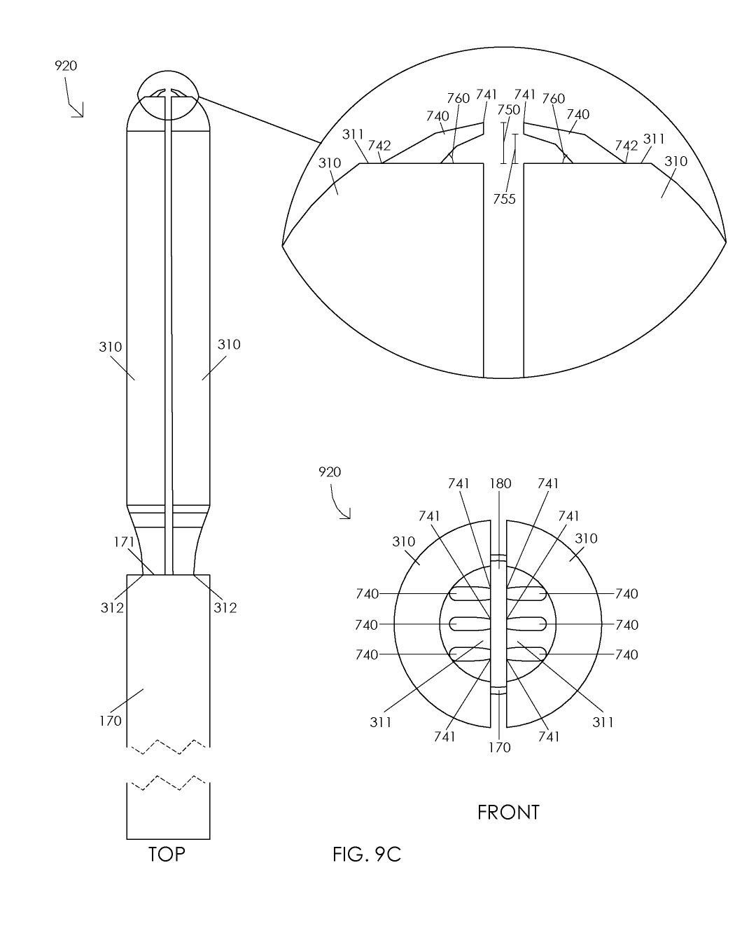

FIGS. 9A, 9B, and 9C are schematic diagrams illustrating a gradual opening of a membrane removing forceps 700. FIG. 9A illustrates a top view and a front view of a closed membrane removing forceps 900. In one or more embodiments, membrane removing forceps 700 may comprise a closed membrane removing forceps 900, e.g., when a first forceps jaw distal end 311 is adjacent to a second forceps jaw distal end 311. Illustratively, membrane removing forceps 700 may comprise a closed membrane removing forceps 900, e.g., when outer hypodermic tube 170 is fully extended over forceps jaws proximal ends 312. In one or more embodiments, membrane removing forceps 700 may comprise a closed membrane removing forceps 900, e.g., when handle 110 is fully compressed.

FIG. 9B illustrates a top view and a front view of a partially open membrane removing forceps 910. In one or more embodiments, a decompression of handle 110 may be configured to gradually open a membrane removing forceps 700, e.g., from a closed membrane removing forceps 900 to a partially open membrane removing forceps 910. Illustratively, a decompression of handle 110 may be configured to retract outer hypodermic tube 170 relative to surgical blank 180, e.g., a decompression of handle 110 may be configured to retract outer hypodermic tube distal end 171 relative to forceps jaws proximal ends 312. In one or more embodiments, a decompression of handle 110 may be configured to gradually separate forceps jaws 310. Illustratively, a decompression of handle 110 may be configured to gradually separate forceps jaws 310 wherein a first forceps jaw distal end 311 contacts a second forceps jaw distal end 311 until all other portions of forceps jaws 310 are separated. In one or more embodiments, a decompression of handle 110 may be configured to separate forceps jaws 310 wherein forceps jaws distal ends 311 are the last portions of forceps jaws 310 to separate.

FIG. 9C illustrates a top view and a front view of a fully open membrane removing forceps 920. Illustratively, a decompression of handle 110 may be configured to gradually open a membrane removing forceps 700, e.g., from a partially open membrane removing forceps 910 to a fully open membrane removing forceps 920. In one or more embodiments, a decompression of handle 110 may be configured to retract outer hypodermic tube 170 relative to surgical blank 180, e.g., a decompression of handle 110 may be configured to retract outer hypodermic tube distal end 171 relative to forceps jaws proximal ends 312. Illustratively, a decompression of handle 110 may be configured to gradually separate forceps jaws 310. In one or more embodiments, a first forceps jaw distal end 311 and a second forceps jaw distal end 311 may be separated by forceps jaw maximum separation distance 315, e.g., when membrane removing forceps 700 comprises a fully open membrane removing forceps 920.

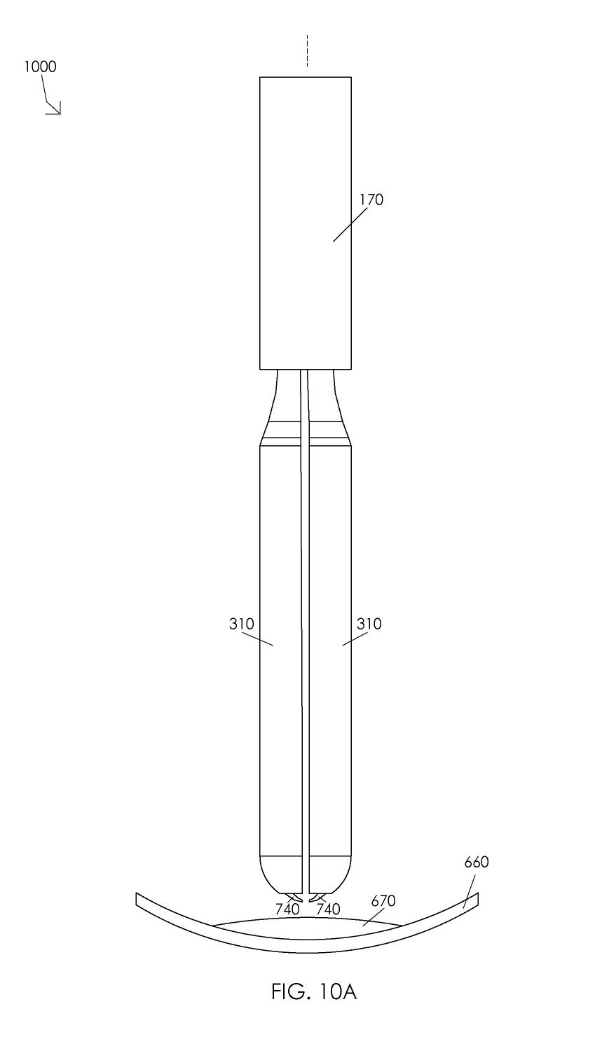

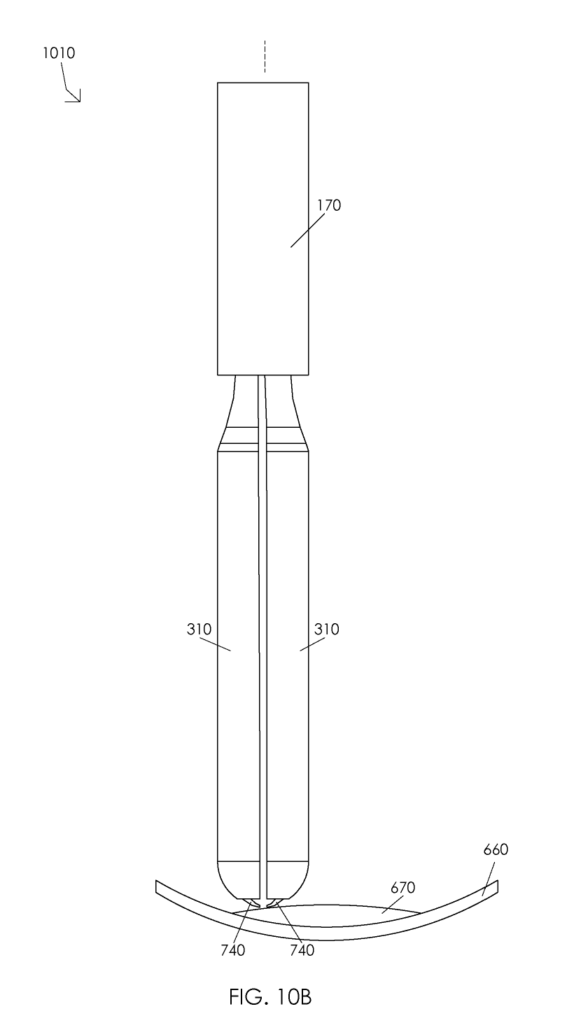

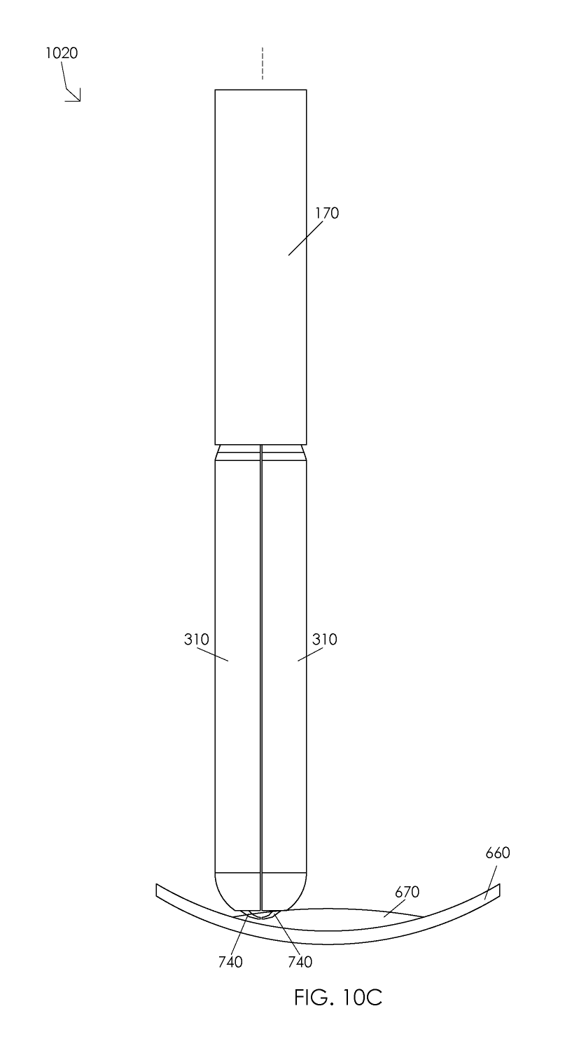

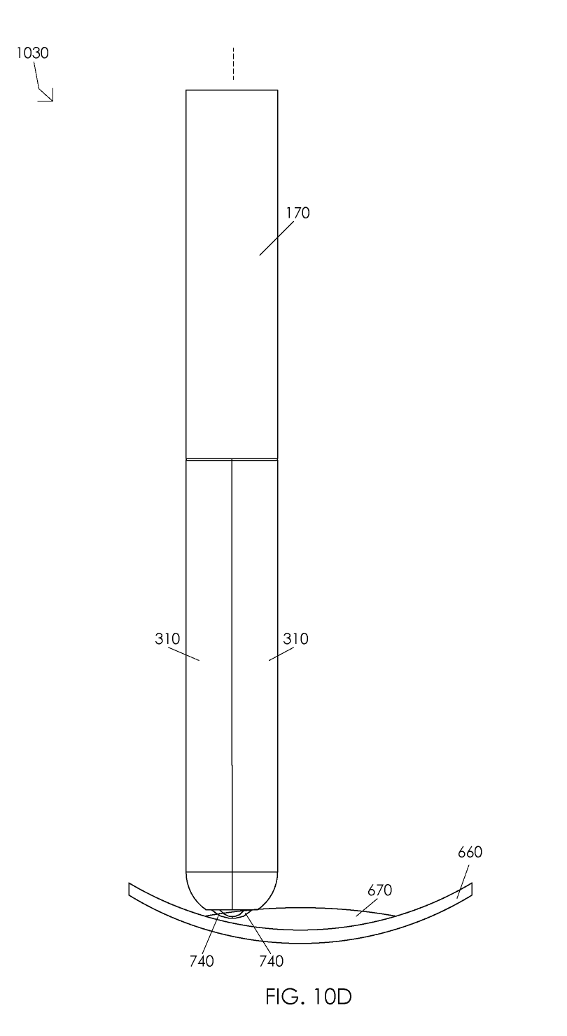

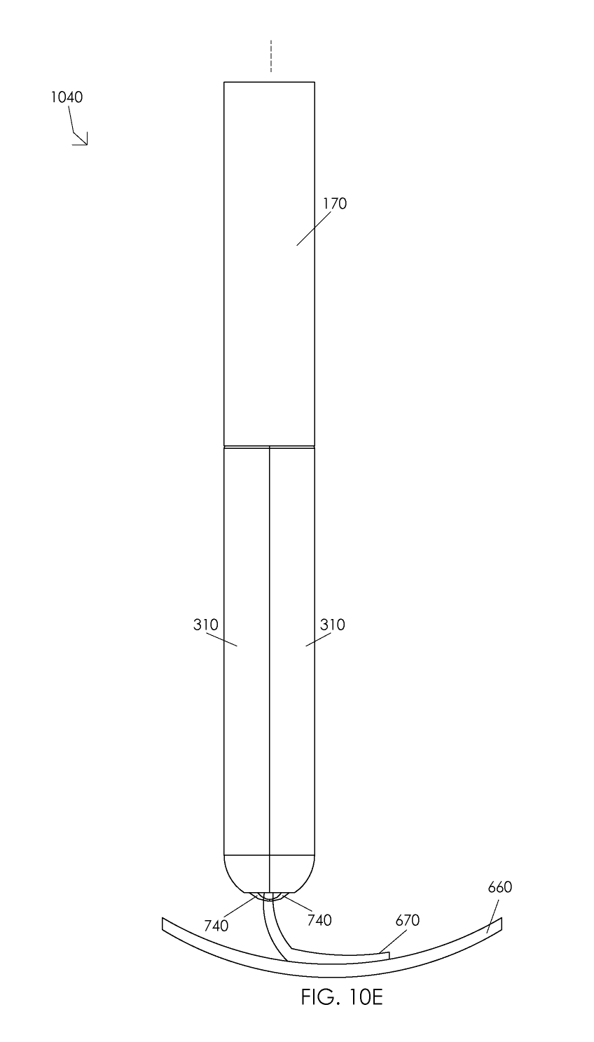

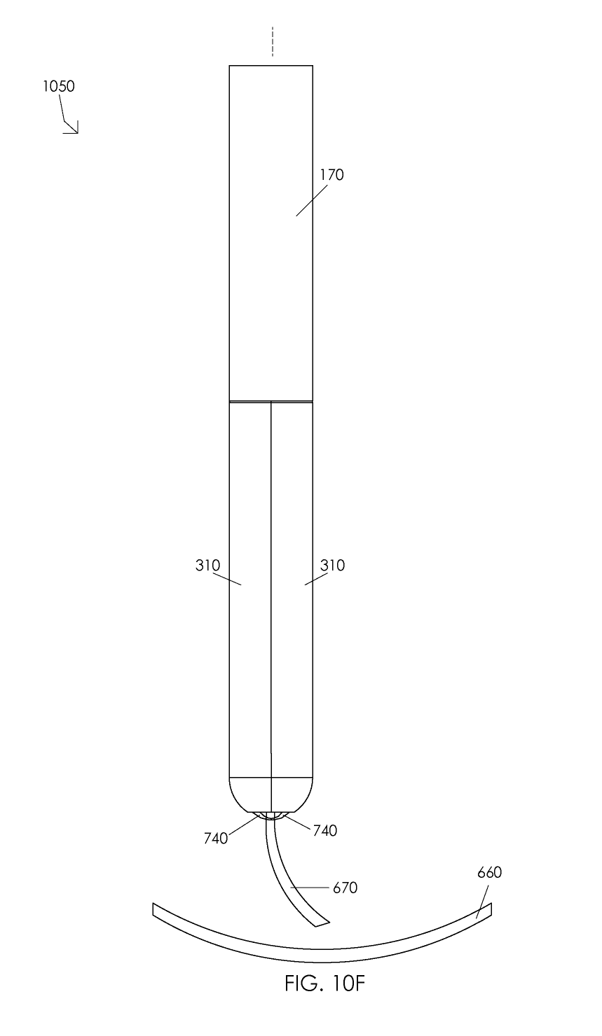

FIGS. 10A, 10B, 10C, 10D, 10E and 10F are schematic diagrams illustrating a membrane removal. FIG. 1 OA illustrates an attached membrane 1000. Illustratively, an attached membrane 1000 may comprise an internal limiting membrane 670 attached to a retina 660. In one or more embodiments, a surgeon may remove an attached membrane 1000 by grasping a portion of a membrane between forceps jaws 310 and peeling the membrane off of an underlying tissue. Illustratively, a surgeon may remove an internal limiting membrane 670 attached to a retina 660 by grasping a portion of internal limiting membrane 670 between forceps jaws 310 and peeling internal limiting membrane 670 off of retina 660. In one or more embodiments, a surgeon may remove an attached membrane 1000 by grasping a portion of a membrane between membrane hooks 740 and peeling the membrane off of an underlying tissue. Illustratively, a surgeon may remove an internal limiting membrane 670 attached to a retina 660 by grasping a portion of internal limiting membrane 670 between membrane hooks 740 and peeling internal limiting membrane 670 off of retina 660.

FIG. 10B illustrates an initial membrane contact 1010. Illustratively, a surgeon may manipulate membrane removing forceps 700 within an eye to cause a contact between a portion of membrane removing forceps 700 and a portion of an attached membrane 1000, e.g., a surgeon may manipulate membrane removing forceps 700 within an eye to cause an initial membrane contract 1010. In one or more embodiments, an initial membrane contact 1010 may comprise a contact between a portion of membrane hook 740 and a portion of a membrane, e.g., an initial membrane contact 1010 may comprise a contact between membrane hook distal end 741 and internal limiting membrane 670.

FIG. 10C illustrates a membrane piercing 1020. Illustratively, a surgeon may manipulate membrane removing forceps 700 within an eye to cause a portion of membrane removing forceps 700 to penetrate into a membrane, e.g., a surgeon may manipulate membrane removing forceps 700 within an eye to penetrate membrane hooks 740 into internal limiting membrane 670. In one or more embodiments, a surgeon may manipulate membrane removing forceps 700 within an eye to cause a portion of membrane removing forceps 700 to penetrate into a membrane without damaging a tissue underlying the membrane, e.g., a surgeon may manipulate membrane removing forceps 700 within an eye to cause membrane hooks 740 to penetrate into internal limiting membrane 670 without damaging retina 660. Illustratively, a surgeon may manipulate membrane removing forceps 700 within an eye to cause a membrane piercing 1020. In one or more embodiments, a membrane piercing 1020 may comprise penetrating a portion of membrane removing forceps 700 into a membrane, e.g., a membrane piercing 1020 may cornprise penetrating membrane hooks 740 in to internal limiting membrane 670. Illustratively, a membrane piercing 1020 may be configured to prevent damage to a tissue underlying a pierced membrane, e.g., one or more properties of membrane removing forceps 700 may be configured to prevent damage to a tissue underlying a pierced membrane. In one or more embodiments, membrane hook outer height 750 may be configured to prevent damage to a tissue underlying a membrane, e.g., membrane hook outer height 750 may be a distance that is less than the average thickness of the membrane. Illustratively, a surface area of forceps jaw distal end 311 may be configured to prevent damage to a tissue underlying a membrane. In one or more embodiments, membrane removing forceps 700 may be configured to perform a membrane piercing 1020 wherein only membrane hooks 740 penetrate a membrane, e.g., membrane removing forceps 700 may be configured to perform a membrane piercing 1020 wherein only membrane hooks 740 penetrate internal limiting membrane 670. Illustratively, a surface area of forceps jaws distal ends 311 may be configured to prevent any portion of membrane removing forceps 700 other than membrane hooks 740 from penetrating a membrane. In one or more embodiments, a ratio of a surface area of each forceps jaw distal end 311 to a surface area of each membrane hook 740 may be in a range of 750.0 to 1500.0, e.g., a ratio of a surface area of each forceps jaw distal end 311 to a surface area of each membrane hook 740 may be 1334.7. Illustratively, a ratio of a surface area of each forceps jaw distal end 311 to a surface area of each membrane hook 740 may be less than 750.0 or greater than 1500.0. In one or more embodiments, a ratio of a surface area of each forceps jaw distal end 311 to a surface area of each membrane hook 740 may large enough to allow membrane hook 740 to penetrate a membrane and prevent forceps jaw distal end 311 from penetrating the membrane. Illustratively, a ratio of a surface area of each forceps jaw distal end 311 to a surface area of each membrane hook 740 may large enough to allow membrane hook 740 to penetrate internal limiting membrane 670 and prevent forceps jaw distal end 311 from penetrating internal limiting membrane 670.

FIG. 10D illustrates a membrane grasping 1030. Illustratively, a membrane grasping 1030 may comprise disposing a portion of a membrane between forceps jaws 310, e.g., a membrane grasping 1030 may comprise disposing a portion of a membrane between forceps jaws distal ends 311. In one or more embodiments, a membrane gasping 1030 may comprise disposing a portion of internal limiting membrane 670 between forceps jaws distal ends 311. Illustratively, a membrane grasping 1030 may comprise disposing a portion of a membrane between membrane hooks 340, e.g., a membrane grasping 1030 may comprise disposing a portion of a membrane between membrane hooks distal ends 741. In one or more embodiments, a membrane grasping 1030 may comprise disposing a portion of internal limiting membrane 670 between membrane hooks distal ends 741. Illustratively, a surgeon may perform a membrane grasping 1030 by compressing handle 110, e.g., a surgeon may perform a membrane grasping 1030 by compressing handle 110 after a membrane piercing 1020. In one or more embodiments, a compression of handle 110 may be configured to extend outer hypodermic tube 170 relative to surgical blank 180, e.g., a compression of handle 110 may be configured to extend outer hypodermic tube distal end 171 over forceps jaws proximal ends 312. Illustratively, a compression of handle 110 may be configured to decrease a distance between a first membrane hook distal end 741 and a second membrane hook distal end 741. In one or more embodiments, decreasing a distance between a first membrane hook distal end 741 and a second membrane hook distal end 741 may be configured to perform a membrane grasping 1030.

FIG. 10E illustrates a partially removed membrane 1040. Illustratively, a partially removed membrane 1040 may comprise a membrane partially separated from an underlying tissue, e.g., a partially removed membrane 1040 may comprise an internal limiting membrane 670 partially separated from a retina 660. In one or more embodiments, a surgeon may peel a membrane apart from an underlying tissue by grasping the membrane with membrane removing forceps 700 and pulling the membrane apart from the underlying tissue, e.g., a surgeon may peel a membrane apart from an underlying tissue by performing a membrane grasping 1030 and pulling the membrane apart from the underlying tissue. Illustratively, a surgeon may peel a membrane apart from an underlying tissue by grasping the membrane with membrane removing forceps 700 and pulling the membrane apart from the underlying tissue until the membrane comprises a partially removed membrane 1040. In one or more embodiments, a surgeon may peel a membrane apart from an underlying tissue by grasping the membrane with membrane hooks 740 and pulling the membrane apart from the underlying tissue until the membrane comprises a partially removed membrane 1040. Illustratively, a surgeon may peel internal limiting membrane 670 apart from an underlying retina 660 by grasping internal limiting membrane 670 with membrane removing forceps 700 and pulling internal limiting membrane 670 apart from retina 660 until internal limiting membrane 670 comprises a partially removed membrane 1040. In one or more embodiments, a surgeon may peel internal limiting membrane 670 apart from an underlying retina 660 by grasping internal limiting membrane 670 with membrane hooks 740 and pulling internal limiting membrane 670 apart from retina 660 until internal limiting membrane 670 comprises a partially removed membrane 1040.

FIG. 10F illustrates a fully removed membrane 1050. Illustratively, a fully removed membrane 1050 may comprise a membrane completely separated from an underlying tissue, e.g., a fully removed membrane 1050 may comprise an internal limiting membrane 670 completely separated from retina 660. In one or more embodiments, a surgeon may peel a membrane apart from an underlying tissue by grasping the membrane with membrane removing forceps 700 and pulling the membrane apart from the underlying tissue until the membrane comprises a fully removed membrane 1050. Illustratively, a surgeon may peel a membrane apart from an underlying tissue by grasping the membrane with membrane hooks 740 and pulling the membrane apart from the underlying tissue until the membrane comprises a fully removed membrane 1050. In one or more embodiments, a surgeon may peel a membrane apart from an underlying tissue by grasping the membrane with membrane hooks 740 and pulling the membrane apart from the underlying tissue until the membrane comprises a partially removed membrane 1040, e.g., a surgeon may then the grasp the membrane with forceps jaws 310 and peel the membrane apart from the underlying tissue until the membrane comprises a fully removed membrane 1050. Illustratively, a surgeon may continue to peel a partially removed membrane 1040 apart from an underlying tissue until the membrane comprises a fully removed membrane 1050. In one or more embodiments, a surgeon may peel internal limiting membrane 670 apart from retina 660 by grasping internal limiting membrane 670 with membrane removing forceps 700 and pulling internal limiting membrane 670 apart from retina 660 until internal limiting membrane 670 comprises a fully removed membrane 1050. Illustratively, a surgeon may peel internal limiting membrane 670 apart from retina 660 by grasping internal limiting membrane 670 with membrane hooks 740 and pulling internal limiting membrane 670 apart from retina 660 until internal limiting membrane 670 comprises a fully removed membrane 1050. In one or more embodiments, a surgeon may peel internal limiting membrane 670 apart from retina 660 by grasping internal limiting membrane 670 with membrane hooks 740 and pulling internal limiting membrane 670 apart from retina 660 until internal limiting membrane 670 comprises a partially removed membrane 1040, e.g., a surgeon may then the grasp internal limiting membrane 670 with forceps jaws 310 and peel internal limiting membrane 670 apart from retina 660 until internal limiting membrane 670 comprises a fully removed membrane 1050. Illustratively, a surgeon may continue to peel a partially removed membrane 1040 apart from retina 660 until internal limiting membrane 670 comprises a fully removed membrane 1050.

The foregoing description has been directed to particular embodiments of this invention. It will be apparent; however, that other variations and modifications may be made to the described embodiments, with the attainment of some or all of their advantages. Specifically, it should be noted that the principles of the present invention may be implemented in any system. Furthermore, while this description has been written in terms of a surgical instrument, the teachings of the present invention are equally suitable to any systems where the functionality may be employed. Therefore, it is the object of the appended claims to cover all such variations and modifications as come within the true spirit and scope of the invention.

What is claimed is:

1. An instrument comprising:
a handle having a handle distal end and a handle proximal end;
a surgical blank having a surgical blank distal end and a surgical blank proximal end wherein at least a portion of the surgical blank is disposed within the handle;
a first forceps jaw of the surgical blank having a first forceps jaw distal end and a first forceps jaw proximal end;
a first membrane hook of the first forceps jaw having a first membrane hook distal end and a first membrane hook proximal end, the first membrane hook having an outer height in a range of 0.25 to 3.0 micrometers;
a second forceps jaw of the surgical blank having a second forceps jaw distal end and a second forceps jaw proximal end;
a second membrane hook of the second forceps jaw having a second membrane hook distal end and a second membrane hook proximal end, the second membrane hook having an outer height in a range of 0.25 to 3.0 micrometers; and
an outer hypodermic tube having an outer hypodermic tube distal end and an outer hypodermic tube proximal end, the outer hypodermic disposed over a portion of the surgical blank wherein a compression of the handle is configured to decrease a distance between first forceps jaw distal end and second forceps jaw distal end.

2. The instrument of claim 1 wherein the first forceps jaw distal end has a surface area in a range of 0.03 to 0.15 square millimeters.

3. The instrument of claim 2 wherein the surface area of the first forceps jaw distal end is 0.065 square millimeters.

4. The instrument of claim 1 further comprising:
a first contour angle of the first forceps jaw, the first contour angle in a range of 60.0 to 80.0 degrees.

5. The instrument of claim 4 wherein the first contour angle is 72.3 degrees.

6. The instrument of claim 1 further comprising:
a second contour angle of the first forceps jaw, the second contour angle in a range of 95.0 to 120.0 degrees.

7. The instrument of claim 6 wherein the second contour angle is 107.0 degrees.

8. The instrument of claim 1 wherein the first membrane hook has a surface area in a range of 25.0 to 75.0 square micrometers.

9. The instrument of claim 8 wherein the surface area of the first membrane hook is 48.7 square micrometers.

10. The instrument of claim 1 wherein the outer height of the first membrane hook is 1.25 micrometers.

11. The instrument of claim 1 further comprising:
an inner height of the first membrane hook, the inner height in a range of 0.1 to 2.9 micrometers.

12. The instrument of claim 11 wherein the inner height of the first membrane hook is 1.0 micrometers.

13. The instrument of claim 1 further comprising:
a membrane hook angle of the first membrane hook, the membrane hook angle in a range of 5.0 to 20.0 degrees.

14. The instrument of claim 13 where the membrane hook angle of the first membrave hook is 10.0 degrees.

15. The instrument of claim 1 wherein a ratio of a surface area of the first forceps jaw distal end to a surface area of the first membrane hook is in a range of 750.0 to 1500.0.

16. The instrument of claim 15 wherein the ratio of the surface area of the first forceps jaw distal end to the surface area of the first membrane hook is 1334.7.

17. The instrument of claim 1 further comprising:
a third membrane hook of the second forceps jaw having a third membrane hook distal end and a third membrane hook proximal end, the third membrane hook having an outer height in a range of 0.25 to 3.0 micrometers.

18. The instrument of claim 17 further comprising:
a fourth membrane hook of the second forceps jaw having a fourth membrane hook distal end and a fourth membrane hook proximal end, the forth membrane hook having an outer height in a range of 0.25 to 3.0 micrometers.

19. The instrument of claim 18 further comprising:
a fifth membrane hook of the second forceps jaw having a fifth membrane hook distal end and a fifth membrane hook proximal end, the fifth membrane hook having an outer height in a range of 0.25 to 3.0 micrometers.

20. The instrument of claim 19 further comprising:
a sixth membrane hook of the second forceps jaw having a sixth membrane hook distal end and a sixth membrane hook proximal end, the sixth membrane hook having an outer height in a range of 0.25 to 3.0 micrometers.
